SEQUENCE LISTING (106 sequences)

SEQ ID NO: 1 - RNA, 40 nt, St. Louis encephalitis virus
  misc_feature 37, 38, 39: n = A,U,C or G
gaaaucuguuuccucuccgcucaccgacgcgaacaunnnc

SEQ ID NO: 2 - RNA, 40 nt, Japanese encephalitis virus
caacgauacuaagccaagaaguucacacagauaaacuucu

SEQ ID NO: 3 - RNA, 40 nt, Murray Valley encephalitis virus
aaacaauacugagaucggaagcucacgcagaugaacgucu

SEQ ID NO: 4 - RNA, 40 nt, West Nile virus
aaacacuacuaaguuugucagcucacacaggcgaacuacu

SEQ ID NO: 5 - RNA, 40 nt, West Nile virus
uugcagaccaaugcaccucaauuagcacacaggauuuacu

SEQ ID NO: 6 - RNA, 40 nt, Dengue type 2 virus
caaagaaucugucuuugucgguccacguagacuaacaacu

SEQ ID NO: 7 - RNA, 40 nt, Hepatitis C virus
gugauucaugguggagugucgcccccaucagggggcuggc

SEQ ID NO: 8 - RNA, 40 nt, Polio virus
gugggccucugggguggguacaaccccagagcuguuuuaa

SEQ ID NO: 9 - RNA, 40 nt, Human enterovirus A
gugggcccuguggguggguacaacccacaggcuguuuuaa

SEQ ID NO: 10 - RNA, 40 nt, Human enterovirus B
aaugggccugugggugggaacaacccacaggcuguuuuaa

SEQ ID NO: 11 - RNA, 40 nt, Human enterovirus C
gugggccucuggggugggagcaaccccagagcuguuuuaa

SEQ ID NO: 12 - RNA, 40 nt, Human enterovirus D
gugggccucuggggugggaacaaccccagagcuguuuuaa

SEQ ID NO: 13 - RNA, 40 nt, Human enterovirus E
agaguacaacacccagugggccuguugggugggaacacuc

SEQ ID NO: 14 - RNA, 40 nt, Bovine enterovirus
gugggccccagggguggguacaacccccaggcuguuuuaa

SEQ ID NO: 15 - RNA, 40 nt, Human rhinovirus 89
auggguggagugagugggaacaacccacucccaguuuuaa

SEQ ID NO: 16 - RNA, 40 nt, Human rhinovirus B
ccaaugggucgaauggugggauacccauccgcuguuuuaa

SEQ ID NO: 17 - RNA, 40 nt, Foot-and-mouth disease virus
guuggcgugcuagagaugagacccuagugcccccuuucaa

SEQ ID NO: 18 - RNA, 40 nt, Hepatitis A virus
ccaagagggacuccggaaauucccggagaccccucuugaa

SEQ ID NO: 19 - RNA, 40 nt, Feline calicivirus
gaagcucagaguuugagacauugucucaaauuucuuuuac

SEQ ID NO: 20 - RNA, 40 nt, Canine calicivirus
gagcucgagagagcgauggcagaagccauuucucauuaac

SEQ ID NO: 21 - RNA, 40 nt, Porcine enteric calicivirus
gcccaauaggcaacggacggcaauuagccaucacgaucac

SEQ ID NO: 22 - RNA, 40 nt, Calicivirus strain NB
aagaaaagugaaagucacuaucucucuauaauuaaaucac

SEQ ID NO: 23 - RNA, 40 nt, Norwalk virus
agcaguaggaacgacgucuuuugacgccaucaucauucac

SEQ ID NO: 24 - RNA, 40 nt, Hepatitis E virus
ugaugccaggagccuuaauaaacugaugggccuccauggc

SEQ ID NO: 25 - RNA, 40 nt, Rubella virus
augggaaugggaguccuaagcgagguccgauagcuuccau

SEQ ID NO: 26 - RNA, 40 nt, SARS coronavirus TOR2
agguugguuggcuuuuccuggguagguaaaaaccuaauau

SEQ ID NO: 27 - RNA, 40 nt, Porcine epidemic diarrhea virus
aaaagagcuaacuauccguagauagaaaaucuuuuuaagu

SEQ ID NO: 28 - RNA, 40 nt, Transmissible gastroenteritis virus
aagagauauagccacgcuacacucacuuuacuuuaaaagu

SEQ ID NO: 29 - RNA, 40 nt, Bovine coronavirus
ucagugaagcgggaugcacgcacgcaaaucgcucgcaauc

SEQ ID NO: 30 - RNA, 40 nt, Human coronavirus 229E
aagcaacuuuucuaucuguagauagauaagguacuuaagu

SEQ ID NO: 31 - RNA, 40 nt, Murine hepatitis virus
agaguugagaggguacguacggacgccaaucacucuuaua

SEQ ID NO: 32 - DNA, 20 bp, Artificial Sequence
  synthetic oligomer
gnngatgttcgcgtcggtga

SEQ ID NO: 33 - DNA, 21 bp, Artificial Sequence
  synthetic oligomer
gtcggtgagcggagaggaaac

SEQ ID NO: 34 - DNA, 20 bp, Artificial Sequence
  synthetic oligomer
agaagtttatctgtgtgaac

SEQ ID NO: 35 - DNA, 22 bp, Artificial Sequence
  synthetic oligomer
ctgtgtgaacttcttggcttag

SEQ ID NO: 36 - DNA, 20 bp, Artificial Sequence
  synthetic oligomer
agacgttcatctgcgtgagc

SEQ ID NO: 37 - DNA, 21 bp, Artificial Sequence
  synthetic oligomer
gttcatctgcgtgagcttccg

SEQ ID NO: 38 - DNA, 22 bp, Artificial Sequence
  synthetic oligomer
agtagttcgcctgtgtgagctg

SEQ ID NO: 39 - DNA, 21 bp, Artificial Sequence
  synthetic oligomer
gtgagctgacaaacttagtag

SEQ ID NO: 40 - DNA, 22 bp, Artificial Sequence
  synthetic oligomer
agtaaatcctgtgtgctaattg

SEQ ID NO: 41 - DNA, 20 bp, Artificial Sequence
  synthetic oligomer
gtgctaattgaggtgcattg

SEQ ID NO: 42 - DNA, 22 bp, Artificial Sequence
  synthetic oligomer
agttgttagtctacgtggaccg

SEQ ID NO: 43 - DNA, 21 bp, Artificial Sequence
  synthetic oligomer
tacgtggaccgacaaagacag

SEQ ID NO: 44 - DNA, 16 bp, Artificial Sequence
  synthetic oligomer
gccagccccctgatgg

SEQ ID NO: 45 - DNA, 22 bp, Artificial Sequence
  synthetic oligomer
atgggggcgacactccaccatg

SEQ ID NO: 46 - DNA, 20 bp, Artificial Sequence
  synthetic oligomer
ttaaaacagctctggggttg

SEQ ID NO: 47 - DNA, 19 bp, Artificial Sequence
  synthetic oligomer
gttgtacccaccccagagg

SEQ ID NO: 48 - DNA, 20 bp, Artificial Sequence
  synthetic oligomer
ttaaaacagcctgtgggttg

SEQ ID NO: 49 - DNA, 19 bp, Artificial Sequence
  synthetic oligomer
gttgtacccacccacaggg

SEQ ID NO: 50 - DNA, 20 bp, Artificial Sequence
  synthetic oligomer
ttaaaacagcctgtgggttg

SEQ ID NO: 51 - DNA, 18 bp, Artificial Sequence
  synthetic oligomer
gttgttcccacccacagg

SEQ ID NO: 52 - DNA, 20 bp, Artificial Sequence
  synthetic oligomer
ttaaaacagctctggggttg

SEQ ID NO: 53 - DNA, 19 bp, Artificial Sequence
  synthetic oligomer
gttgctcccaccccagagg

SEQ ID NO: 54 - DNA, 20 bp, Artificial Sequence
  synthetic oligomer
ttaaaacagctctggggttg

SEQ ID NO: 55 - DNA, 18 bp, Artificial Sequence
  synthetic oligomer
ttgttcccaccccagagg

SEQ ID NO: 56 - DNA, 20 bp, Artificial Sequence
  synthetic oligomer
gagtgttcccacccaacagg

SEQ ID NO: 57 - DNA, 20 bp, Artificial Sequence
  synthetic oligomer
aacaggcccactgggtgttg

SEQ ID NO: 58 - DNA, 20 bp, Artificial Sequence
  synthetic oligomer
ttaaaacagcctgggggttg

SEQ ID NO: 59 - DNA, 19 bp, Artificial Sequence
  synthetic oligomer
gttgtacccacccctgggg

SEQ ID NO: 60 - DNA, 20 bp, Artificial Sequence
  synthetic oligomer
ttaaaactgggagtgggttg

SEQ ID NO: 61 - DNA, 20 bp, Artificial Sequence
  synthetic oligomer
gttgttcccactcactccac

SEQ ID NO: 62 - DNA, 21 bp, Artificial Sequence
  synthetic oligomer
ttaaaacagcggatgggtatc

SEQ ID NO: 63 - DNA, 20 bp, Artificial Sequence
  synthetic oligomer
gatgggtatcccaccattcg

SEQ ID NO: 64 - DNA, 19 bp, Artificial Sequence
  synthetic oligomer
ttgaaagggggcactaggg

SEQ ID NO: 65 - DNA, 20 bp, Artificial Sequence
  synthetic oligomer
agggtctcatctctagcacg

SEQ ID NO: 66 - DNA, 19 bp, Artificial Sequence
  synthetic oligomer
ttcaagaggggtctccggg

SEQ ID NO: 67 - DNA, 21 bp, Artificial Sequence
  synthetic oligomer
gaatttccggagtccctcttg

SEQ ID NO: 68 - DNA, 22 bp, Artificial Sequence
  synthetic oligomer
gtaaaagaaatttgagacaatg

SEQ ID NO: 69 - DNA, 19 bp, Artificial Sequence
  synthetic oligomer
gtctcaaactctgagcttc

SEQ ID NO: 70 - DNA, 21 bp, Artificial Sequence
  synthetic oligomer
gttaatgagaaatggcttctg

SEQ ID NO: 71 - DNA, 22 bp, Artificial Sequence
  synthetic oligomer
cttctgccatcgctctctcgag

SEQ ID NO: 72 - DNA, 20 bp, Artificial Sequence
  synthetic oligomer
gtgatcgtgatggctaattg

SEQ ID NO: 73 - DNA, 22 bp, Artificial Sequence
  synthetic oligomer
aattgccgtccgttgcctattg

SEQ ID NO: 74 - DNA, 23 bp, Artificial Sequence
  synthetic oligomer
gtgatttaattatagagagatag

SEQ ID NO: 75 - DNA, 22 bp, Artificial Sequence
  synthetic oligomer
ttatagagagatagtgactttc

SEQ ID NO: 76 - DNA, 23 bp, Artificial Sequence
  synthetic oligomer
gtgaatgatgatggcgtcaaaag

SEQ ID NO: 77 - DNA, 21 bp, Artificial Sequence
  synthetic oligomer
caaaagacgtcgttcctactg

SEQ ID NO: 78 - DNA, 18 bp, Artificial Sequence
  synthetic oligomer
gccatggaggcccatcag

SEQ ID NO: 79 - DNA, 22 bp, Artificial Sequence
  synthetic oligomer
atcagtttattaaggctcctgg

SEQ ID NO: 80 - DNA, 20 bp, Artificial Sequence
  synthetic oligomer
atggaagctatcggacctcg

SEQ ID NO: 81 - DNA, 22 bp, Artificial Sequence
  synthetic oligomer
tatcggacctcgcttaggactc

SEQ ID NO: 82 - DNA, 23 bp, Artificial Sequence
  synthetic oligomer
atattaggtttttacctacccag

SEQ ID NO: 83 - DNA, 21 bp, Artificial Sequence
  synthetic oligomer
acccaggaaaagccaaccaac

SEQ ID NO: 84 - DNA, 24 bp, Artificial Sequence
  synthetic oligomer
acttaaaaagattttctatctacg

SEQ ID NO: 85 - DNA, 22 bp, Artificial Sequence
  synthetic oligomer
ttttctatctacgtacggatag

SEQ ID NO: 86 - DNA, 21 bp, Artificial Sequence
  synthetic oligomer
acttttaaagtaaagtgagtg

SEQ ID NO: 87 - DNA, 21 bp, Artificial Sequence
  synthetic oligomer
gtaaagtgagtggtagcgtgg

SEQ ID NO: 88 - DNA, 22 bp, Artificial Sequence
  synthetic oligomer
gattgcgagcgatttgcgtgcg

SEQ ID NO: 89 - DNA, 22 bp, Artificial Sequence
  synthetic oligomer
gtgcgtgcatcccgcttcactg

SEQ ID NO: 90 - DNA, 24 bp, Artificial Sequence
  synthetic oligomer
cttaagtaccttatctatctacag

SEQ ID NO: 91 - DNA, 19 bp, Artificial Sequence
  synthetic oligomer
tctacagatagaaaagttg

SEQ ID NO: 92 - DNA, 21 bp, Artificial Sequence
  synthetic oligomer
tataagagtgattggcgtccg

SEQ ID NO: 93 - DNA, 22 bp, Artificial Sequence
  synthetic oligomer
tccgtacgtaccctctcaactc

SEQ ID NO: 94 - DNA, 14 bp, Artificial Sequence
  target sequence based on virus sequence
agtagttcgcctgt

SEQ ID NO: 95 - DNA, 12 bp, Artificial Sequence
  target sequence based on virus sequence
ctgacaaactta

SEQ ID NO: 96 - DNA, 14 bp, Artificial Sequence
  target sequence based on virus sequence
tcgcctgtgtgagc

SEQ ID NO: 97 - DNA, 14 bp, Artificial Sequence
  target sequence based on virus sequence
agtagttcaaactt

SEQ ID NO: 98 - DNA, 13 bp, Artificial Sequence
  target sequence based on virus sequence
agtaaatcctgtg

SEQ ID NO: 99 - DNA, 14 bp, Artificial Sequence
  target sequence based on virus sequence
ctgtgtgctaattg

SEQ ID NO: 100 - DNA, 15 bp, Artificial Sequence
  target sequence based on virus sequence
aatcctgtgtgctaa

SEQ ID NO: 101 - DNA, 14 bp, Artificial Sequence
  target sequence based on virus sequence
agtaaatcaattga

SEQ ID NO: 102 - DNA, 14 bp, Artificial Sequence
  target sequence based on virus sequence
taattgaggtgcat

SEQ ID NO: 103 - DNA, 14 bp, Artificial Sequence
  target sequence based on virus sequence
tgggggcgacactc

SEQ ID NO: 104 - DNA, 16 bp, Artificial Sequence
  scramble control sequence
cgcgacccctgcgatg

SEQ ID NO: 105 - DNA, 20 bp, Artificial Sequence
  control sequence
agtctcgacttgctacctca

SEQ ID NO: 106 - RNA, 380 nt, Hepatitis C virus
gccagcccccugaugggggcgacacuccaccaugaaucacuccccugugaggaacuacugucuucacgcagaaagcgucuagccauggcguuaguaugagugucgugcagccuccaggacccccccucccgggagagccauaguggucugcggaaccggugaguacaccggaauugccaggacgaccggguccuuucuuggauaaacccgcucagaugccuggagauuugggcgugcccccgcaagacugcuagccgaguaguguugggucgcgaaaggccuugugguacugccugauagggugcuugcgagugccccgggaggucucguagaccgugcaccaugagcacgaauccuaaaccucaaagaaaaaccaaacc

(19) United States
(12) Patent Application Publication
(10) Pub. No.: US 2008/0311556 A1
(43) Pub. Date: Dec. 18, 2008

(54) Sense Antiviral Compound and Method for Treating Ssrna Viral Infection

(76) Inventor: Patrick L. Iversen, Corvallis, OR (US)

Correspondence Address: King & Spalding LLP, P.O. Box 889, Belmont, CA, 94002-0889, US

(21) Appl. No.: 10/567,470

(22) PCT Filed: Aug. 6, 2004
(86) PCT No.: PCT/US2004/025401

§ 371 Date: Nov. 30, 2006

Related U.S. Application Data
(60) Provisional application No. 60/493,990, filed on Aug. 7, 2003.

(51) Int. Cl. C12Q 1/70; C07H 21/02; C12N 15/00

(52) U.S. Cl. 435/5; 536/24.5; 435/455

(57) ABSTRACT

The invention provides sense antiviral compounds and methods of their use in inhibition of growth of viruses of the Flaviviridae, Picornoviridae, Caliciviridae, Togaviridae, Coronaviridae families and hepatitis E virus in the treatment of a viral infection. The sense antiviral compounds are substantially uncharged morpholino oligonucleotides having a sequence of (12-40) subunits, including at least (12) subunits having a targeting sequence that is complementary to a region associated with stem-loop secondary structure within the 3′-terminal end (40) bases of the negative-sense RNA strand of the virus.

### FIELD OF THE INVENTION

This invention relates to sense oligonucleotide compounds for use in treating a flavivirus, picornavirus, calicivirus, togavirus, coronavirus and hepatitis E virus infection, antiviral treatment methods employing the compounds, and methods for monitoring binding of sense oligonucleotides to a negative-strand viral genome target site.

### REFERENCES

The following references are related to the background or the invention.
Banerjee, R. and A. Dasgupta (2001). “Interaction of picornavirus 2C polypeptide with the viral negative-strand RNA.” J Gen Virol 82(Pt 11): 2621-7.
Banerjee, R. and A. Dasgupta (2001). “Specific interaction of hepatitis C virus protease/helicase NS3 with the 3′-terminal sequences of viral positive- and negative-strand RNA.” J Virol 75(4): 1708-21.
Banerjee, R., A. Echeverri, et al. (1997). “Poliovirus-encoded 2C polypeptide specifically binds to the 3′-terminal sequences of viral negative-strand RNA.” J Virol 71(12): 9570-8.
Banerjee, R., W. Tsai, et al. (2001). “Interaction of poliovirus-encoded 2C/2BC polypeptides with the 3′ terminus negative-strand cloverleaf requires an intact stem-loop b.” Virology 280(1): 41-51.
Blommers, M. J., U. Pieles, et al. (1994). “An approach to the structure determination of nucleic acid analogues hybridized to RNA. NMR studies of a duplex between 2′-OMe RNA and an oligonucleotide containing a single amide backbone modification.” Nucleic Acids Res 22(20): 4187-94.
Cross, C. W., J. S. Rice, et al. (1997). “Solution structure of an RNA×DNA hybrid duplex containing a 3′-thioformacetal linker and an RNA A-tract.” Biochemistry 36(14): 4096-107.
Gait, M. J., A. S. Jones, et al. (1974). “Synthetic-analogues of polynucleotides XII. Synthesis of thymidine derivatives containing an oxyacetamido- or an oxyformamido-linkage instead of a phosphodiester group.” J Chem Soc [Perkin 1] 0(14): 1684-6.
Holland, J. (1993). Emerging Virus. S. S. Morse. New York and Oxford, Oxford University Press: 203-218.
Lesnikowski, Z. J., M. Jaworska, et al. (1990). “Octa(thymidine methanephosphonates) of partially defined stereochemistry: synthesis and effect of chirality at phosphorus on binding to pentadecadeoxyriboadenylic acid.” Nucleic Acids Res 18(8): 2109-15.
Mertes, M. P. and E. A. Coats (1969). “Synthesis of carbonate analogs of dinucleosides. 3′-Thymidinyl 5′-thymidinyl carbonate, 3′-thymidinyl 5′-(5-fluoro-2′-deoxyuridinyl)carbonate, and 3′-(5-fluoro-2′-deoxyuridinyl) 5′-thymidinyl carbonate.” J Med Chem 12(1): 154-7.
Miller, P. S. (1993). Antisense Research Applications. S. T. Crooke and B. Lebleu. Boca Raton, CRC Press: 189.
Moulton, H. M., M. H. Nelson, et al. (2004). “Cellular uptake of antisense morpholino oligomers conjugated to arginine-rich peptides.” Bioconjug Chem 15(2): 290-9.
Murray, R. and e. al. (1998). Medical Microbiology. St. Louis, Mo., Mosby Press: 542-543.
O'Ryan, M. (1992). Clinical Virology Manual. S. Spector and G. Lancz. New York, Elsevier Science: 361-196.
Pardigon, N., E. Lenches, et al. (1993). “Multiple binding sites for cellular proteins in the 3′ end of Sindbis alphavirus minus-sense RNA.” J Virol 67(8): 5003-11.
Pardigon, N. and J. H. Strauss (1992). “Cellular proteins bind to the 3° end of Sindbis virus minus-strand RNA.” J Virol 66(2): 1007-15.
Paul, A. V. (2002). Possible unifying mechanism of picornavirus genome replication. Molecular Biology of Picornaviruses. B. L. Semler and E. Wimmer. Washington, D.C., ASM Press: 227-246.
Roehl, H. H., T. B. Parsley, et al. (1997). “Processing of a cellular polypeptide by 3CD proteinase is required for poliovirus ribonucleoprotein complex formation.” J Virol 71(1): 578-85.
Roehl, H. H. and B. L. Semler (1995). “Poliovirus infection enhances the formation of two ribonucleoprotein complexes at the 3′ end of viral negative-strand RNA.” J Virol 69(5): 2954-61.
Smith, A. W., D. E. Skilling, et al. (1998). “Calicivirus emergence from ocean reservoirs: zoonotic and interspecies movements.” Emerg Infect Dis 4(1): 13-20.
Wu, G. Y. and C. H. Wu (1992). “Specific inhibition of hepatitis B viral gene expression in vitro by targeted antisense oligonucleotides.” J Biol Chem 267(18): 12436-9.
Xu, W. Y. (1991). “Viral haemorrhagic disease of rabbits in the People's Republic of China: epidemiology and virus characterisation.” Rev Sci Tech 10(2): 393-408.

### BACKGROUND OF THE INVENTION

Single-stranded RNA (ssRNA) viruses cause many diseases in wildlife, domestic animals and humans. These viruses are genetically and antigenically diverse, exhibiting broad tissue tropisms and a wide pathogenic potential. The incubation periods of some of the most pathogenic viruses, e.g. the caliciviruses, are very short. Viral replication and expression of virulence factors may overwhelm early defense mechanisms (Xu 1991) and cause acute and severe symptoms.

There are no specific treatment regimes for many viral infections. The infection may be serotype specific and natural immunity is often brief or absent (Murray and al. 1998). Immunization against these virulent viruses is impractical because of the diverse serotypes. RNA virus replicative processes lack effective genetic repair mechanisms, and current estimates of RNA virus replicative error rates are such that each genomic replication can be expected to produce one to ten errors, thus generating a high number of variants (Holland 1993). Often, the serotypes show no cross protection such that infection with any one serotype does not protect against infection with another. For example, vaccines against the vesivirus genus of the caliciviruses would have to provide protection against over 40 different neutralizing serotypes (Smith, Skilling et al. 1998) and vaccines for the other genera of the Caliciviridae are expected to have the same limitations.

Thus, there remains a need for an effective antiviral therapy in several virus families, including small, single-stranded, positive-sense RNA viruses in the flavivirus, picornavirus, calicivirus, togavirus, and coronavirus families.

### SUMMARY OF THE INVENTION

The invention includes, in one aspect, an oligonucleotide analog compound for use in inhibiting replication in mammalian host cells of an RNA virus having a single-stranded, positive-sense RNA genome and selected from from the Flaviviridae, Picornoviridae, Caliciviridae, Togaviridae, or Coronaviridae families and hepatitis E virus. The compound is characterized by:

(i) a nuclease-resistant backbone,

(ii) capable of uptake by mammalian host cells,

(iii) containing between 12-40 nucleotide bases,

(iv) having a targeting sequence of at least 12 subunits that is complementary to a region associated with stem-loop secondary structure within the 3′-terminal end 40 bases of the negative-sense RNA strand of the virus, and

(v) capable of forming with the negative-strand viral ssRNA genome, a heteroduplex structure having a Tm of dissociation of at least 45° C. and disruption of the stem-loop secondary structure.

An exemplary compound is composed of morpholino subunits linked by uncharged, phosphorus-containing intersubunit linkages, joining a morpholino nitrogen of one subunit to a 5′ exocyclic carbon of an adjacent subunit. The compound may have phosphorodiamidate linkages, such as in the structure

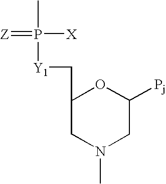

where Y₁=O, Z=O, Pj is a purine or pyrimidine base-pairing moiety effective to bind, by base-specific hydrogen bonding, to a base in a polynucleotide, and X is alkyl, alkoxy, thioalkoxy, or alkyl amino. In a preferred compound, X═NR₂, where each R is independently hydrogen or methyl.

The heteroduplex structure formed may have a Tm of greater than 45° C., e.g., 50-80° C., and may be actively taken up by the cells.

For treatment of the virus given below, the targeting sequence is complementary to a region associated with stem-loop secondary structure within one of the following sequences:

(i) SEQ ID NO. 1, for St Louis encephalitis virus;
(ii) SEQ ID NO. 2, for Japanese encephalitis virus;
(iii) SEQ ID NO. 3, for a Murray Valley encephalitis virus;
(iv) SEQ ID NO. 4, for a West Nile fever virus;
(v) SEQ ID NO. 5, for a Yellow fever virus
(vi) SEQ ID NO. 6, for a Dengue type 2 virus; and
(vii) SEQ ID NO. 7, for a Hepatitis C virus.

For treatment of a picornovirus, the targeting sequence is complementary to a region associated with stem-loop secondary structure within one of the following sequences:

(i) SEQ ID NO. 8, for a polio virus of the Mahoney and Sabin strains;
(ii) SEQ ID NO. 9, for a Human enterovirus A;
(iii) SEQ ID NO. 10, for a Human enterovirus B;
(iv) SEQ ID NO. 11, for a Human enterovirus C;
(v) SEQ ID NO. 12, for a Human enterovirus D;
(vi) SEQ ID NO. 13, for a Human enterovirus E;
(vii) SEQ ID NO. 14, for a Bovine enterovirus;
(viii) SEQ ID NO. 15, for Human rhinovirus 89;
(ix) SEQ ID NO. 16, for Human rhinovirus B;
(x) SEQ ID NO. 17, for Foot-and-mouth disease virus; and
(xi) SEQ ID NO. 18, for a hepatitis A virus,

For treatment of a calici virus, the targeting sequence is complementary to a region associated with stem-loop secondary structure within one of the following sequences:

### (i) SEQ ID NO. 19, for Feline Calicivirus;

### (ii) SEQ ID NO. 20, for Canine Calicivirus;

(iii) SEQ ID NO. 21, for Porcine enteric calicivirus;
(iv) SEQ ID NO. 22, for Calicivirus strain NB; and
(v) SEQ ID NO. 23, for Norwalk virus.

For treatment of Hepatitis E virus, the targeting sequence is complementary to a region associated with stem-loop secondary structure within the sequence identified as SEQ ID NO: 24.

For treatment of a Togaviridae, Rubella virus, the targeting sequence is complementary to a region associated with stem-loop secondary structure within the sequence identified as SEQ ID NO: 25.

For treatment of a Coronaviridae, the targeting sequence is complementary to a region associated with stem-loop secondary structure within one of the following sequences:

(i) SEQ ID NO. 26, for SARS coronavirus TOR2;
(ii) SEQ ID NO. 27, for Porcine epidemic diarrhea virus;
(iii) SEQ ID NO. 28, for Transmissible gastroenteritis virus;
(iv) SEQ ID NO. 29, for Bovine coronavirus;
(v) SEQ ID NO. 30, for Human coronavirus 229E. and
(vi) SEQ ID NO. 31, for Murine hepatitis virus.

Also disclosed is a complex formed between the compound and the negative strand of the viral genome, by hybridization of the analog compound with the complementary-sequence at the 3′-end region of the negative-strand RNA of the virus.

In another aspect, the invention is directed to a method of inhibiting, in a mammalian host cell, replication of an RNA virus from the Flaviviridae, Picomoviridae, Caliciviridae, Togaviridae, Coronaviridae families and hepatitis E virus, where the virus has a single-stranded, positive-sense genome. In practicing the method, the host cells are exposed to the above oligonucleotide analog compound, thus to form within the cells, a heteroduplex structure (i) composed of the negative sense strand of the virus and the oligonucleotide compound, and (ii) characterized by a Tm of dissociation of at least 45° C. and disruption of stem-loop secondary structure in the 3′-end 40 base region of the negative strand RNA. The compound may have various of the embodiments noted above.

Also forming part of the invention is a method of confirming the presence of an effective interaction between a picornavirus, calicivirus, togavirus, coronavirus, hepatitis E virus, or flavivirus infecting a mammalian subject, and an uncharged morpholino sense oligonucleotide analog compound against the infecting virus. This method involves first administering to the subject, an uncharged morpholino sense analog compound of the type described above. At a selected time after this administering, a sample of a body fluid is obtained from the subject. The sample is assayed for the presence of a nuclease-resistant heteroduplex composed of the sense oligonucleotide complexed with a complementary-sequence 3′-end region of the negative-strand RNA of the virus.

These and other objects and features of the invention will be more fully appreciated when the following detailed description of the invention is read in conjunction with the accompanying figures.

### BRIEF DESCRIPTION OF THE DRAWING

FIGS. 1A-1G show the backbone structures of various oligonucleotide analogs with uncharged backbones;

FIGS. 2A-2D show the repeating subunit segment of exemplary morpholino oligonucleotides, designated 2A-2D;

FIGS. 3A-3E are schematic diagrams of genomes of exemplary viruses and viral target sites;

FIGS. 4A-4D show examples of predicted secondary structures of 3′ end terminal minus-strand regions for exemplary viruses; and

FIG. 5 represents an immunoblot of cellular extracts prepared from hepatitis C virus-infected cells treated with a sense oligomer (SEQ ID NO. 13) directed to the 3′-end-terminus of the minus-strand RNA and appropriate controls.

FIG. 6 MHC-induced cytopathic effects 48 hours post infection under various treatment regimens, in accordance with the invention.

### DETAILED DESCRIPTION OF THE INVENTION

### I. Definitions

The terms below, as used herein, have the following meanings, unless indicated otherwise:

The terms “oligonucleotide analog” or “oligonucleotide analog compound” refers to oligonucleotide having (i) a modified backbone structure, e.g., a backbone other than the standard phosphodiester linkage found in natural oligo- and polynucleotides, and (ii) optionally, modified sugar moieties, e.g., morpholino moieties rather than ribose or deoxyribose moieties. The analog supports bases capable of hydrogen bonding by Watson-Crick base pairing to standard polynucleotide bases, where the analog backbone presents the bases in a manner to permit such hydrogen bonding in a sequence-specific fashion between the oligonucleotide analog molecule and bases in a standard polynucleotide (e.g., single-stranded RNA or single-stranded DNA). Preferred analogs are those having a substantially uncharged, phosphorus-containing backbone.

A substantially uncharged, phosphorus containing backbone in an oligonucleotide analog is one in which a majority of the subunit linkages, e.g., between 60-100%, are uncharged at physiological pH, and contain a single phosphorous atom. The analog contains between 8 and 40 subunits, typically about 8-25 subunits, and preferably about 12 to 25 subunits. The analog may have exact sequence complementarity to the target sequence or near complementarity, as defined below.

A “subunit” of an oligonucleotide analog refers to one nucleotide (or nucleotide analog) unit of the analog. The term may refer to the nucleotide unit with or without the attached intersubunit linkage, although, when referring to a “charged subunit”, the charge typically resides within the intersubunit linkage (e.g. a phosphate or phosphorothioate linkage).

A “morpholino oligonucleotide analog” is an oligonucleotide analog composed of morpholino subunit structures of the form shown in FIGS. 2A-2D, where (i) the structures are linked together by phosphorus-containing linkages, one to three atoms long, joining the morpholino nitrogen of one subunit to the 5′ exocyclic carbon of an adjacent subunit, and (ii) Pᵢ and Pⱼ are purine or pyrimidine base-pairing moieties effective to bind, by base-specific hydrogen bonding, to a base in a polynucleotide. The purine or pyrimidine base-pairing moiety is typically adenine, cytosine, guanine, uracil or thymine. The synthesis, structures, and binding characteristics of morpholino oligomers are detailed in U.S. Pat. Nos. 5,698,685, 5,217,866, 5,142,047, 5,034,506, 5,166,315, 5,521,063, and 5,506,337, all of which are incorporated herein by reference.

The subunit and linkage shown in FIG. 2B are used for six-atom repeating-unit backbones (where the six atoms include: a morpholino nitrogen, the connected phosphorus atom, the atom (usually oxygen) linking the phosphorus atom to the 5′ exocyclic carbon, the 5′ exocyclic carbon, and two carbon atoms of the next morpholino ring). In these structures, the atom Y₁ linking the 5′ exocyclic morpholino carbon to the phosphorus group may be sulfur, nitrogen, carbon or, preferably, oxygen. The X moiety pendant from the phosphorus is any stable group which does not interfere with base-specific hydrogen bonding. Preferred X groups include fluoro, alkyl, alkoxy, thioalkoxy, and alkyl amino, including cyclic amines, all of which can be variously substituted, as long as base-specific bonding is not disrupted. Alkyl, alkoxy and thioalkoxy preferably include 1-6 carbon atoms. Alkyl amino preferably refers to lower alkyl (C₁ to C₆) substitution, and cyclic amines are preferably 5- to 7-membered nitrogen heterocycles optionally containing 1-2 additional heteroatoms selected from oxygen, nitrogen, and sulfur. Z is sulfur or oxygen, and is preferably oxygen.

A preferred morpholino oligomer is a phosphorodiamidate-linked morpholino oligomer, referred to herein as a PMO. Such oligomers are composed of morpholino subunit structures such as shown in FIG. 2B, where X═NH₂, NHR, or NR₂ (where R is lower alkyl, preferably methyl), Y═O, and Z=O, and Pᵢ and Pⱼ are purine or pyrimidine base-pairing moieties effective to bind, by base-specific hydrogen bonding, to a base in a polynucleotide. Also preferred are structures having an alternate phosphorodiamidate linkage, where, in FIG. 2B, X=lower alkoxy, such as methoxy or ethoxy, Y═NH or NR, where R is lower alkyl, and Z=O.

The term “substituted”, particularly with respect to an alkyl, alkoxy, thioalkoxy, or alkylamino group, refers to replacement of a hydrogen atom on carbon with a heteroatom-containing substituent, such as, for example, halogen, hydroxy, alkoxy, thiol, alkylthio, amino, alkylamino, imino, oxo (keto), nitro, cyano, or various acids or esters such as carboxylic, sulfonic, or phosphonic. It may also refer to replacement of a hydrogen atom on a heteroatom (such as an amine hydrogen) with an alkyl, carbonyl or other carbon containing group.

As used herein, the term “target”, relative to the viral genomic RNA, refers to a viral genomic RNA, and specifically, to a region associated with stem-loop secondary structure within the 3′-terminal end 40 bases of the negative-sense RNA strand of a ssRNA virus described herein.

The term “target sequence” refers to a portion of the target RNA against which the oligonucleotide analog is directed, that is, the sequence to which the oligonucleotide analog will hybridize by Watson-Crick base pairing of a complementary sequence. As will be seen, the target sequence may be a contiguous region of the viral negative strand RNA, or may be composed of complementary fragments of both the 5′ and 3′ sequences involved in secondary structure.

The term “targeting sequence” is the sequence in the oligonucleotide analog that is complementary (meaning, in addition, substantially complementary) to the target sequence in the RNA genome. The entire sequence, or only a portion of, the analog compound may be complementary to the target sequence. For example, in an analog having 20 bases, only 12-14 may be targeting sequences. Typically, the targeting sequence is formed of contiguous bases in the analog, but may alternatively be formed of non-contiguous sequences that when placed together, e.g., from opposite ends of the analog, constitute sequence that spans the target sequence. As will be seen, the target and targeting sequences are selected such that binding of the analog to a region within the 3′-terminal end 40 bases of the negative-sense RNA strand of the virus acts to disrupt secondary structure in the viral RNA, particularly, the most 3′ stem loop structure, in this region.

Target and targeting sequences are described as “complementary” to one another when hybridization occurs in an antiparallel configuration. A targeting may have “near” or “substantial” complementarity to the target sequence and still function for the purpose of the present invention, that is, still be “complementary.” Preferably, the oligonucleotide analog compounds employed in the present invention have at most one mismatch with the target sequence out of 10 nucleotides, and preferably at most one mismatch out of 20. Alternatively, the sense oligomers employed have at least 90% sequence homology, and preferably at least 95% sequence homology, with the exemplary positive-strand targeting sequences as designated herein.

An oligonucleotide analog “specifically hybridizes” to a target polynucleotide if the oligomer hybridizes to the target under physiological conditions, with a Tₘ greater than 45° C., preferably at least 50° C., and typically 60° C.-80° C. or higher. Such hybridization preferably corresponds to stringent hybridization conditions. At a given ionic strength and pH, the Tₘ is the temperature at which 50% of a target sequence hybridizes to a complementary polynucleotide. Again, such hybridization may occur with “near” or “substantial” complementary of the antisense oligomer to the target sequence, as well as with exact complementarity.

A “nuclease-resistant” oligomeric molecule (oligomer) refers to one whose backbone is substantially resistant to nuclease cleavage, in non-hybridized or hybridized form; by common extracellular and intracellular nucleases in the body; that is, the oligomer shows little or no nuclease cleavage under normal nuclease conditions in the body to which the oligomer is exposed.

A “heteroduplex” refers to a duplex between an oligonculeotide analog and the complementary portion of a target RNA. A “nuclease-resistant heteroduplex” refers to a heteroduplex formed by the binding of an antisense oligomer to its complementary target, such that the heteroduplex is substantially resistant to in vivo degradation by intracellular and extracellular nucleases, such as RNAseH, which are capable of cutting double-stranded RNA/RNA or RNA/DNA complexes.

A “base-specific intracellular binding event involving a target RNA” refers to the specific binding of an oligonucleotide analog to a target RNA sequence inside a cell. The base specificity of such binding is sequence specific. For example, a single-stranded polynucleotide can specifically bind to a single-stranded polynucleotide that is complementary in sequence.

An “effective amount” of an antisense oligomer, targeted against an infecting ssRNA virus, is an amount effective to reduce the rate of replication of the infecting virus, and/or viral load, and/or symptoms associated with the viral infection.

As used herein, the term “body fluid” encompasses a variety of sample types obtained from a subject including, urine, saliva, plasma, blood, spinal fluid, or other sample of biological origin, such as skin cells or dermal debris, and may refer to cells or cell fragments suspended therein, or the liquid medium and its solutes.

The term “relative amount” is used where a comparison is made between a test measurement and a control measurement. The relative amount of a reagent forming a complex in a reaction is the amount reacting with a test specimen, compared with the amount reacting with a control specimen. The control specimen may be run separately in the same assay, or it may be part of the same sample (for example, normal tissue surrounding a malignant area in a tissue section).

“Treatment” of an individual or a cell is any type of intervention provided as a means to alter the natural course of the individual or cell. Treatment includes, but is not limited to, administration of the oligonucleotide analog compound, and may be performed either prophylactically, or subsequent to the initiation of a pathologic event or contact with an etiologic agent. The related term “improved therapeutic outcome” relative to a patient diagnosed as infected with a particular virus, refers to a slowing or diminution in the growth of virus, or viral load, or detectable symptoms associated with infection by that particular virus.

An agent is “actively taken up by mammalian cells” when the agent can enter the cell by a mechanism other than passive diffusion across the cell membrane. The agent may be transported, for example, by “active transport”, referring to transport of agents across a mammalian cell membrane by e.g. an ATP-dependent transport mechanism, or by “facilitated transport”, referring to transport of antisense agents across the cell membrane by a transport mechanism that requires binding of the agent to a transport protein, which then facilitates passage of the bound agent across the membrane. For both active and facilitated transport, the oligonucleotide analog preferably has a substantially uncharged backbone, as defined below. Alternatively, the antisense compound may be formulated in a complexed form, such as an agent having an anionic backbone complexed with cationic lipids or liposomes, which can be taken into cells by an endocytotic mechanism. The analog may be conjugated, e.g., at its 5′ or 3′ end, to an arginine rich peptide, e.g., the HIV TAT protein, or polyarginine, to facilitate transport into the target host cell.

### II. Targeted Viruses

The present invention is based on the discovery that effective inhibition of certain classes of single-stranded, positive-sense RNA viruses can be achieved by exposing cells infected with the virus to sense oligonucleotide analog compounds (I) targeted against the 3′ end terminal sequences of the minus-strand (negative-sense) viral RNA strand, and in particular, against target sequences that contribute to stem-loop secondary structure in this region, (ii) having physical and pharmacokinetic features which allow effective interaction between the sense compound and the virus within host cells. In one aspect, the oligomers can be used in treating a mammalian subject infected with the virus.

The invention targets RNA viruses having genomes that are: (i) single stranded, (ii) positive polarity, and (iii) less than 32 kb. The targeted viruses also synthesize a genomic RNA strand with negative polarity, the minus-strand or negative-sense RNA, as the first step in viral RNA replication. In particular, targeted viral families include Flaviviridae, Picornaviridae, Caliciviridae, Togaviridae, Coronaviridae, and Hepatitis E virus. Various physical, morphological, and biological characteristics of each of these five families, and members therein, can be found, for example, in Textbook of Human Virology, R. Belshe, ed., 2^{nd} Edition, Mosby, 1991 and at the Universal Virus Database of the International Committee on Taxonomy of Viruses which can be accessed at (http://www.ncbi.nim.nih.gov/ICTVdb/index.htm). Some of the key biological characteristics of each family are summarized below.

A. Flaviviridae. Members of this family include several serious human pathogens, among them mosquito-borne members of the genus Flavivirus including yellow fever, West Nile fever, Japanese encephalitis, St. Louis encephalitis, Murray Valley encephalitis, and Dengue. The Flaviviridae also includes Hepatitis C virus, a member of the Hepacivirus genus.

Flaviviridae virions are approximately 40 to 50 nm in diameter. The symmetry of the nucleocapsid has not been fully defined. It is known that the Flaviviridae envelope contains only one species of glycoprotein. As yet, no subgenomic messenger RNA nor polyprotein precursors have been detected for members of the Flaviviridae.

B. Picornaviridae. This family, whose members infect both humans and animals, can cause severe paralysis (paralytic poliomyelitis), aspectic meningitis, hepatitis, pleurodynia, myocarditis, skin rashes, and colds; inapparent infection is common. Several medically important members include the poliovirus, hepatitis A virus, rhinovirus, Aphthovirus (foot- and mouth disease virus), human enterovirus, and the coxsackie virus.

Rhinoviruses are recognized as the principle cause of the common cold in humans. Serotypes are designated from 1A to 100. Transmission is primarily by the aerosol route and the virus replicates in the nose.

Like all positive-sense RNA viruses, the genomic RNA of Picornaviruses is infectious; that is, the genomic RNA is able to direct the synthesis of viral proteins directly, without host transcription events.

C. Caliciviridae. The caliciviridae infect both humans and animals. The genus Vesivirus produces disease manifestations in mammals that include epithelial blistering and are suspected of being the cause of animal abortion storms and human hepatitis (non A through E) (Smith et al., 1998a and 1998b). Other genera of the Caliciviridae include the Norwalk-like and Sapporo-like viruses, which together comprise the human calicivirus, and the Lagoviruses, which include rabbit hemorrhagic disease virus, a particularly rapid and deadly virus.

The human caliciviruses are the most common cause of viral diarrhea outbreaks worldwide in adults, as well as being significant pathogens of infants (O'Ryan 1992). There are at least five types of human caliciviruses that inhabit the gastrointestinal tract. The Norwalk virus is a widespread human agent causing acute epidemic gastroenteritis and causes approximately 10% of all outbreaks of gastroenteritis in man (Murray and al. 1998).

Vesiviruses are now emerging from being regarded as somewhat obscure and host specific to being recognized as one of the more versatile groups of viral pathogens known. For example, a single serotype has been shown to infect a diverse group of 16 different species of animals that include a saltwater fish (opal eye), sea lion, swine, and man.

D. Togaviridae. Members of this family include the mosquito-borne viruses which infect both humans and animals. The family includes the genera Alphavirus (sindbis) and Rubivirus (rubella).

E. Hepatitis E-like Viruses. Hepatitis E virus (HEV) was initially described in 1987 and first reported in the U.S. in 1991. The virus was initially described as a member of the Caliciviridae based on the small, single-stranded RNA character. Some still classify HEV as belonging to the Caliciviridae, but it has also been classified as a member of the Togavirus family. It currently has no family classification. Infection appears to be much like hepatitis A viral infection. The disease is an acute viral hepatitis which is apparent about 20 days after initial infection, and the virus may be observed for about 20 days in the serum. Transmission occurs through contaminated water and geographically the virus is restricted to less developed countries.

F. Coronaviridae. Members of this family include human corona viruses that cause 10 to 30% of common colds and other respiratory infections, and murine hepatitis virus. More recently, the viral cause of severe acute respiratory syndrome (SARS) has been identified as a coronavirus.

### III. Viral Target Regions

Single-stranded, positive-sense RNA viruses, like all RNA viruses, are unique in their ability to synthesize RNA on an RNA template. To achieve this task they encode and induce the synthesis of a unique RNA-dependent RNA polymerases (RdRp) and possibly other proteins which bind specifically to the 3′ and 5′ end terminal UTRs of viral RNA. Since viral RNAs are linear molecules, RdRps have to employ unique strategies to initiate de novo RNA replication while retaining the integrity of the 5′ end of their genomes. It is generally accepted that positive-strand (+strand) viral RNA replication proceeds via the following pathway:

+strand RNA→−strand RNA synthesis→RF→+strand RNA synthesis

where “−strand RNA” is negative-sense or minus-strand RNA complementary to the “+strand RNA” and “RF” (replicative form) is double-stranded RNA. The minus-strand RNA is used as a template for replication of multiple copies of positive-strand RNA which is destined for either translation into viral proteins or incorporation into newly formed virions. The ratio of positive to minus-strand RNA in poliovirus-infected cells is approximately 50:1 to 30:1 in Hepatitis C-infected cells, indicating that each minus-strand RNA serves as a template for the synthesis of many positive-strand RNA molecules.

There is evidence that RNA:RdRp interactions require recognition motifs for specific inititiation of minus- and plus-strand RNA synthesis. These recognition motifs are usually contained within conserved stem-loop structures inside the 5′- and 3′-terminal regions. Studies in numerous systems have shown these stem-loop structures (or cis-acting determinants) to be important for viral RNA replication in many positive-strand RNA viruses. Most molecular studies utilizing in vitro systems have investigated the cis-acting elements within the 5′ and 3′ UTRs of positive-strand RNA. The role of the 3′ UTR of negative-strand RNA, possibly together with the 5′ UTR of positive-strand RNA in initiation of positive-strand RNA viral replication by RdRp is not understood. However, poliovirus replication has been studied in some detail and a role for cis-acting elements within the 3′ minus-strand UTR has been proposed (Paul 2002).

Poliovirus is the prototype Picornavirus and its replication mechanism has been studied extensively (Paul 2002). Both viral-encoded (Banerjee, Echeverri et al., 1997; Banerjee and Dasgupta 2001; Banerjee, Tsai et al. 2001) and cellular proteins (Roehl and Semler 1995; Roehl, Parsley et al. 1997) are thought to bind specifically to the 3′ UTR of minus-strand poliovirus RNA. In addition both hepatitis c virus (Banerjee and Dasgupta 2001) and Sindbis virus (Pardigon and Strauss 1992; Pardigon, Lenches et al. 1993) encode proteins that bind specifically to their minus-strand RNA. Although the mechanism remains unknown, the protein:RNA interactions that have been observed may be essential for replication of postitive-strand RNA from the minus-strand template.

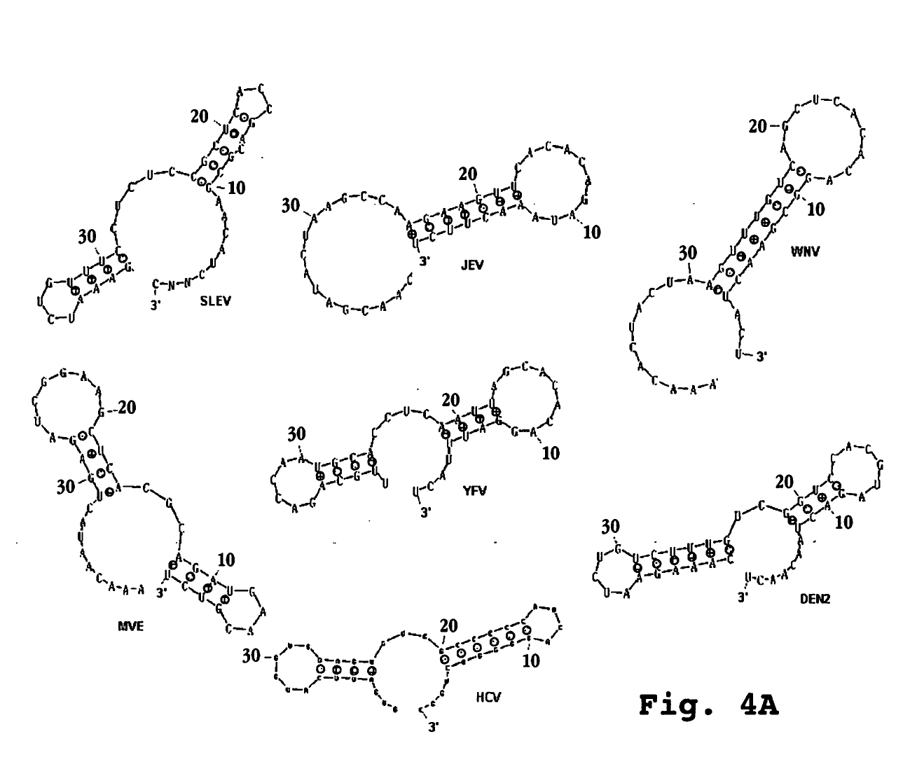

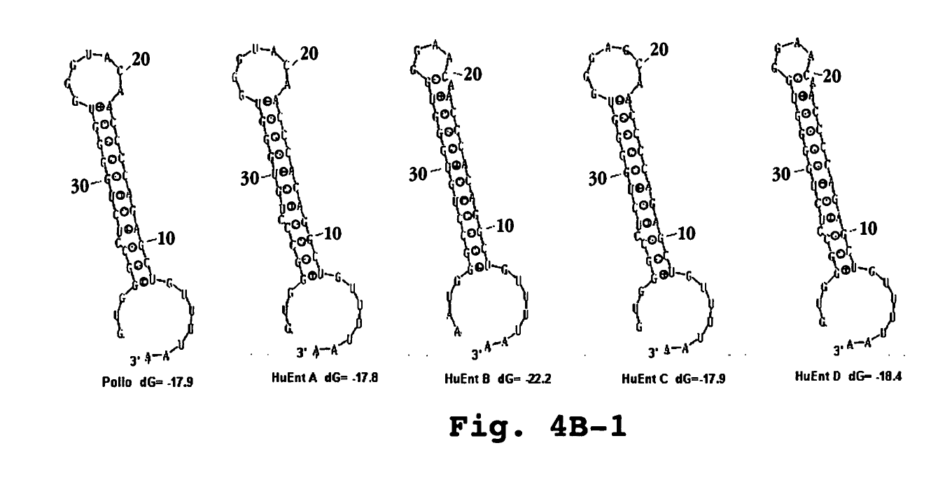

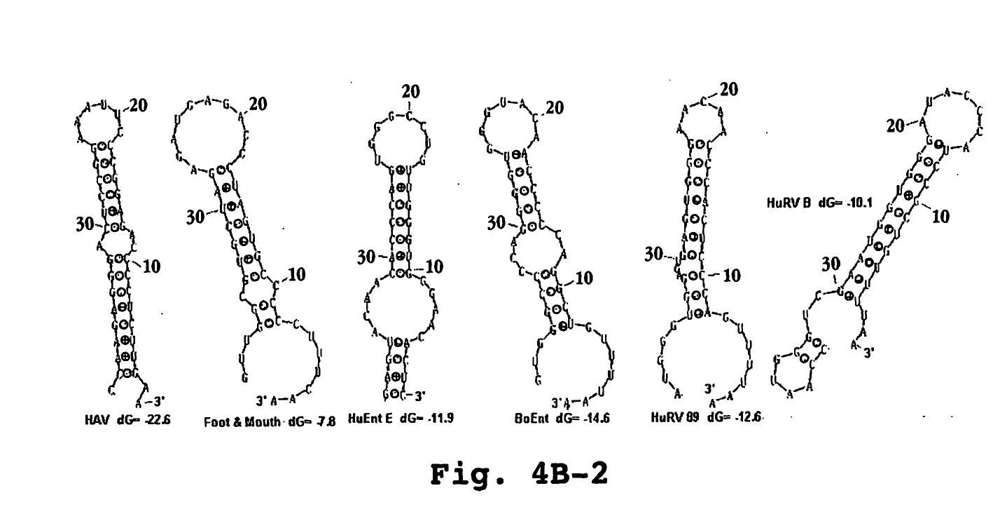

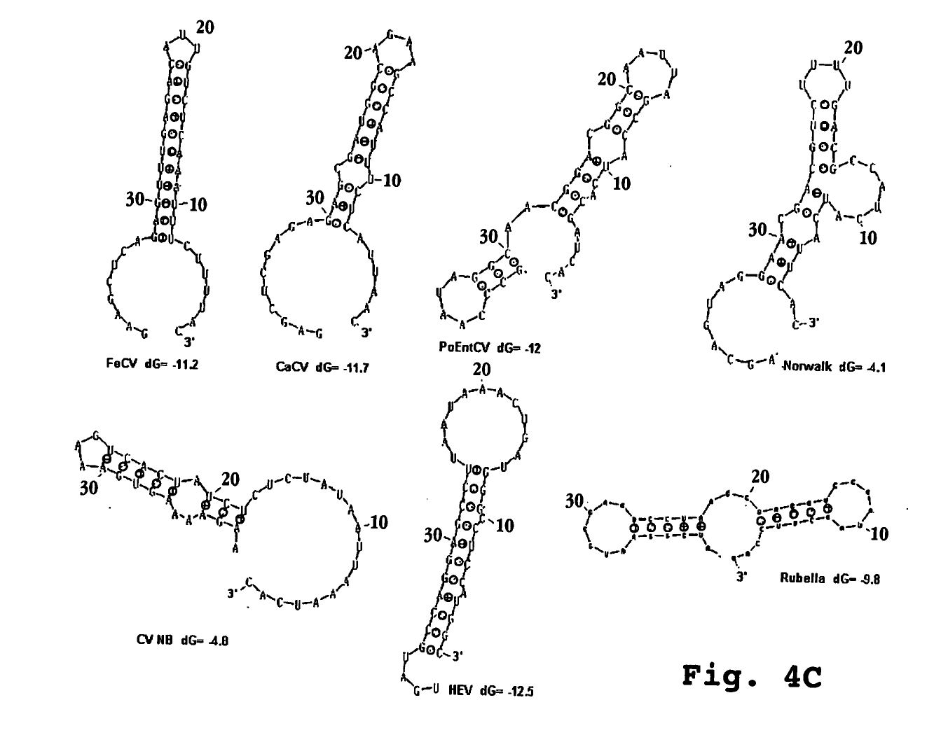

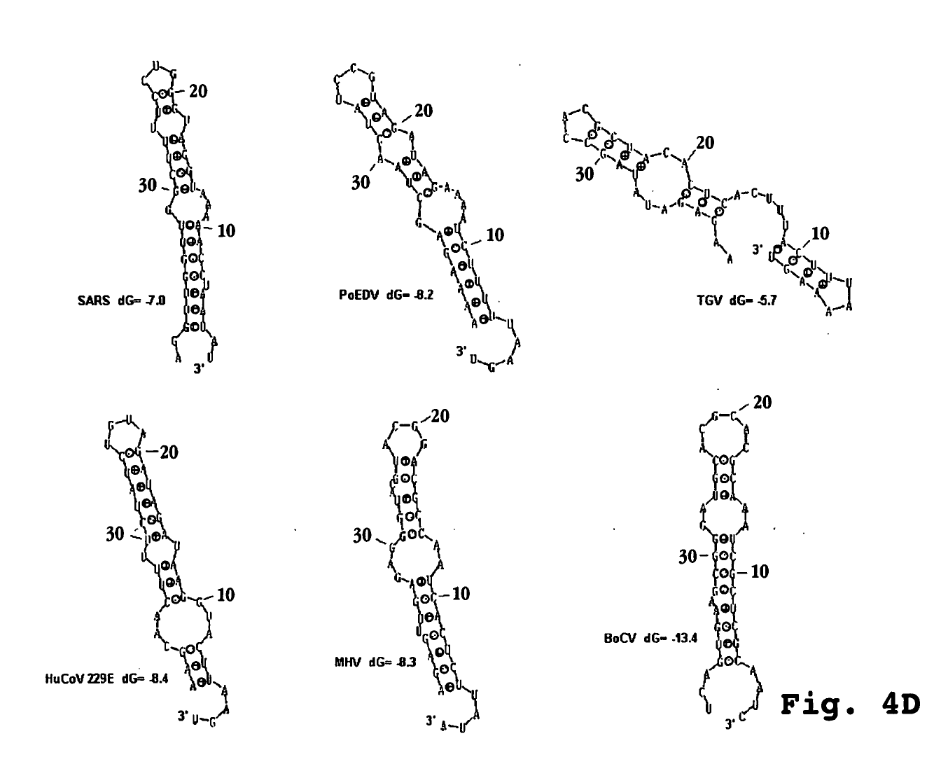

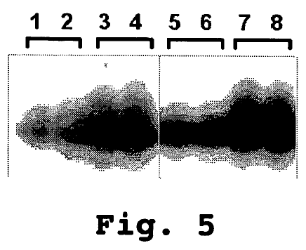

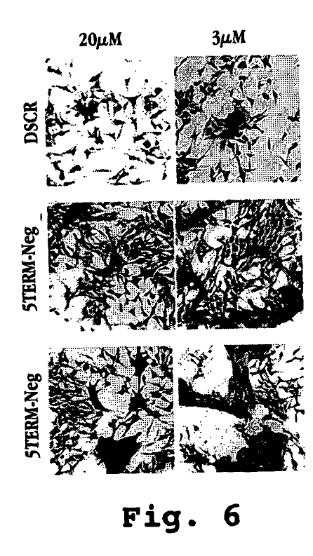

The cis-acting elements for most positive-strand RNA viruses are poorly characterized due to the difficulty in elucidating their structure and function. One experimental tool is to utilize computer-assisted secondary structure predictions which are based on a search for the minimal free energy state of the input RNA sequence. The predicted secondary structures or stem loops of the 3′ end terminal minus-strand RNA from several representative single-stranded, positive-sense RNA viruses are shown in FIGS. 4A-4D. Inhibition of HCV viral replication was discovered by the inventors when sense oligomers were targeted to the 3′ end-terminal minus-strand stem-loop of hepatitis C virus.

Therefore, the preferred target sequences are the 3′ end terminal regions of the minus-strand RNA. These regions include the end-most 40 nucleotides and preferably the terminal 20 nucleotides. The specific target regions include bases that contribute to secondary structure in this region, as indicated in FIGS. 4A-4C. In particular, the targeting sequence contains a sequence of at least 12 bases that are complementary to 3′-end region of the negative strand RNA, and are selected such that hybridization of the compound to the RNA is effective to disrupt stem-loop secondary structure in this region, preferably the 3′-end most stem-loop secondary structure. By way of example, FIGS. 4A-4D shows secondary structure in several 3′-end negative strand viral sequences. These sequences, and sequences for related viruses, are available from well known sources, such as the NCBI Genbank databases. Alternatively, a person skilled in the art can find sequences for many of the subject viruses in the open literature, e.g., by searching for references that disclose sequence information on designated viruses. Once a complete or partial viral sequence is obtained, the 5′ end-terminal sequences of the virus are identified. The general genomic organization of each of the five virus families is discussed below, followed by exemplary target sequences obtained for selected members (genera, species or strains) within each family.

A. Picornaviridae. Typical of the picornaviruses, the human rhinovirus 89 genome (FIG. 3A) is a single molecule of single-stranded, positive-sense, polyadenylated RNA of approximately 7.2 kb. The genome includes a long 618 nucleotide UTR which is located upstream of the first polyprotein, a single ORF, and a VPg (viral genome linked) protein covalently attached to its 5′ end. The ORF is subdivided into two segments, each of which encodes a polyprotein. The first segment encodes a polyprotein that is cleaved subsequently to form viral proteins VP1 to VP4, and the second segment encodes a polyprotein which is the precursor of viral proteins including a protease and a polymerase. The ORF terminates in a polyA termination sequence.

B. Caliciviridae. FIG. 3B shows the genome of a calicivirus; in this case the Norwalk virus. The genome is a single molecule of infectious, single stranded, positive-sense RNA of approximately 7.6 kb. As shown, the genome includes a small UTR upstream of the first open reading frame which is unmodified. The 3′ end of the genome is polyadenylated. The genome includes three open reading frames. The first open reading frame encodes a polyprotein, which is subsequently cleaved to form the viral non-structural proteins including a helicase, a protease, an RNA dependent RNA polymerase, and “VPg”, a protein that becomes bound to the 5′ end of the viral genomic RNA (Clarke and Lambden, 2000). The second open reading frame codes for the single capsid protein, and the third open reading frame codes for what is reported to be a structural protein that is basic in nature and probably able to associate with RNA.

C. Togaviridae. FIG. 3C shows the structure of the genome of a togavirus, in this case, a rubella virus of the Togavirus genus. The genome is a single linear molecule of single-stranded, positive-sense RNA of approximately 9.8 kb, which is infectious. The 5′ end is capped with a 7-methylG molecule and the 3′ end is polyadenylated. Full-length and subgenomic messenger RNAs have been demonstrated, and post translational cleavage of polyproteins occurs during RNA replication. The genome also includes two open reading frames. The first open reading frame encodes a polyprotein which is subsequently cleaved into four functional proteins, nsP1 to nsP4. The second open reading frame encodes the viral capsid protein and three other viral proteins, PE2, 6K and E1.

D. Flaviviridae. FIG. 3D shows the structure of the genome of the hepatitis C virus of the Hepacivirus genus. The HCV genome is a single linear molecule of single-stranded, positive-sense RNA of about 9.6 kb and contains a 341 nucleotide 5′ UTR. The 5′ end is capped with an m⁷ GppAmp molecule, and the 3′ end is not polyadenylated. The genome includes only one open reading frame which encodes a precursor polyprotein separable into six structural and functional proteins.

E. Coronaviridae. FIG. 3E shows the genome structure of human coronavirus 229E. This coronovirus has a large genome of approximately 27.4 kb that is typical for the Coronoviridae and a 292 nucleotide 5′ UTR. The 5′-most ORF of the viral genome is translated into a large polyprotein that is cleaved by viral-encoded proteases to release several nonstructural proteins, including an RdRp and a helicase. These proteins, in turn, are responsible for replicating the viral genome as well as generating nested transcripts that are used in the synthesis of other viral proteins.

GenBank references for exemplary viral nucleic acid sequences representing the 3′ end terminal, minus-strand sequences for the first (most 3′-emd) 40 bases for corresponding viral genomes are listed in Table 1, below. The nucleotide sequence numbers in Table 1 are derived from the Genbank reference for the positive-strand RNA. It will be appreciated that these sequences are only illustrative of other sequences in the five virus families, as may be available from available gene-sequence databases of literature or patent resources. The sequences below, identified as SEQ ID NOs 1-31, are also listed in Table 3 at the end of the specification.

The target sequences in Table 1 are the first 40 bases at the 3′ terminal ends of the minus-strands or negative-sense sequences of the indicated viral RNAs. The sequences shown are in the 5′ to 3′ orientation so the 3′ terminal nucleotide is at the end of the listed sequence. The region within each sequence that is associated with stem-loop secondary structure can be seen from the predicted secondary structures in these sequences, shown in FIGS. 4A-4D.

TABLE 1

Exemplary 3′ End Terminal Viral Nucleic Acid
Target Sequences

| | | | | |
|---|---|---|---|---|
| | | | | SEQ |
| | GenBank | Target | Target Sequence | ID |
| Virus | Acc. No. | Ncts. | (5′ to 3′) | NO. |

| | | | | |
|---|---|---|---|---|
| St. Louis encephalitis | M18929 | 1-40 | GAAAUCUGUUUCCUCUCCGCUC | 1 |
| (SLEV) | | | ACCGACGCGAACAUNNNC | |
| Japanese encephalitis | NC 001437 | 1-40 | CAACGAUACUAAGCCAAGAAGU | 2 |
| (JEV) | | | UCACACAGAUAAACUUCU | |
| Murray Valley | NC 000943 | 1-40 | AAACAAUACUGAGAUCGGAAGC | 3 |
| encephalitis (MVEV) | | | UCACGCAGAUGAACGUCU | |
| West Nile | NC 001563 | 1-40 | AAACACUACUAAGUUUGUCAGC | 4 |
| (WNV) | | | UCACACAGGCGAACUACU | |
| Yellow Fever | NC 002031 | 1-40 | UUGCAGACCAAUGCACCUCAAU | 5 |
| (YFV) | | | UAGCACACAGGAUUUACU | |
| Dengue - Type 2 | M20558 | 1-40 | CAAAGAAUCUGUCUUUGUCGGU | 6 |
| (DEN2) | | | CCACGUAGACUAACAACU | |
| Hepatitis C | NC 004102 | 1-40 | GUGAUUCAUGGUGGAGUGUCGC | 7 |
| (HCV) | | | CCCCAUCAGGGGGCUGGC | |
| Poliovirus-Mahoney | NC 002058 | 1-40 | GUGGGCCUCUGGGGUGGGUACA | 8 |
| strain (Polio) | | | ACCCCAGAGCUGUUUUAA | |
| Human enterovirus A | NC 001612 | 1-40 | GUGGGCCCUGUGGGUGGGUACA | 9 |
| (HuEntA) | | | ACCCACAGGCUGUUUUAA | |
| Human enterovirus B | NC 001472 | 1-40 | AAUGGGCCUGUGGGUGGGAACA | 10 |
| (HuEntB) | | | ACCCACAGGCUGUUUUAA | |
| Human enterovirus C | NC 001428 | 1-40 | GUGGGCCUCUGGGGUGGGAGCA | 11 |
| (HuEntC) | | | ACCCCAGAGCUGUUUUAA | |
| Human enterovirus D | NC 001430 | 1-40 | GUGGGCCUCUGGGGUGGGAACA | 12 |
| (HuEntD) | | | ACCCCAGAGCUGUUUUAA | |
| Human enterovirus E | NC 003988 | 1-40 | AGAGUACAACACCCAGUGGGCC | 13 |
| (HuEntE) | | | UGUUGGGUGGGAACACUC | |
| Bovine enterovirus | NC 001859 | 1-40 | GUGGGCCCCAGGGGUGGGUACA | 14 |
| (BoEnt) | | | ACCCCCAGGCUGUUUUAA | |
| Human rhinovirus 89 | NC 001617 | 1-40 | AUGGGUGGAGUGAGUGGGAACA | 15 |
| (HuRV89) | | | ACCCACUCCCAGUUUUAA | |
| Human rhinovirus B | NC 001490 | 1-40 | CCAAUGGGUCGAAUGGUGGGAU | 16 |
| (HuRVB) | | | ACCCAUCCGCUGUUUUAA | |
| Foot-and-mouth | NC 004004 | 1-40 | GUUGGCGUGCUAGAGAUGAGAC | 17 |
| disease | | | CCUAGUGCCCCCUUUCAA | |
| (Foot and Mouth) | | | | |
| Hepatitis A | NC 001489 | 1-40 | CCAAGAGGGACUCCGGAAAUUC | 18 |
| (HAV) | | | CCGGAGACCCCUCUUGAA | |
| Feline calicivirus | NC 001481 | 1-40 | GAAGCUCAGAGUUUGAGACAUU | 19 |
| (FeCV) | | | GUCUCAAAUUUCUUUUAC | |
| Canine calicivirus | NC 004542 | 1-40 | GAGCUCGAGAGAGCGAUGGCAG | 20 |
| (CaCV) | | | AAGCCAUUUCUCAUUAAC | |
| Porcine enteric | NC 000940 | 1-40 | GCCCAAUAGGCAACGGACGGCA | 21 |
| calicivirus | | | AUUAGCCAUCACGAUCAC | |
| (PoEntCV) | | | | |
| Calicivirus strain NB | NC 004064 | 1-40 | AAGAAAAGUGAAAGUCACUAUC | 22 |
| (CVNB) | | | UCUCUAUAAUUAAAUCAC | |
| Norwalk | NC 001959 | 1-40 | AGCAGUAGGAACGACGUCUUUU | 23 |
| (Norwalk) | | | GACGCCAUCAUCAUUCAC | |
| Hepatitis E | NC 001434 | 1-40 | UGAUGCCAGGAGCCUUAAUAAA | 24 |
| (HEV) | | | CUGAUGGGCCUCCAUGGC | |
| Rubella | NC 001545 | 1-40 | AUGGGAAUGGGAGUCCUAAGCG | 25 |
| (Rubella) | | | AGGUCCGAUAGCUUCCAU | |
| SARS coronavirus | NC 004718 | 1-40 | AGGUUGGUUGGCUUUUCCUGGG | 26 |
| TOR2 | | | UAGGUAAAAACCUAAUAU | |
| (SARS) | | | | |
| Porcine epidemic | NC 003436 | 1-40 | AAAAGAGCUAACUAUCCGUAGA | 27 |
| diarrhea | | | UAGAAAAUCUUUUUAAGU | |
| (PoEDV) | | | | |
| Transmissible | NC 002306 | 1-40 | AAGAGAUAUAGCCACGCUACAC | 28 |
| gastroenteritis | | | UCACUUUACUUUAAAAGU | |
| (TGV) | | | | |
| Bovine coronavirus | NC 003045 | 1-40 | UCAGUGAAGCGGGAUGCACGCA | 29 |
| (BoCoV) | | | CGCAAAUCGCUCGCAAUC | |
| Human coronavirus | NC 002645 | 1-40 | AAGCAACUUUUCUAUCUGUAGA | 30 |
| 2290E | | | UAGAUAAGGUACUUAAGU | |
| (HuCoV229E) | | | | |
| Murine Hepatitis | NC 001846 | 1-40 | AGAGUUGAGAGGGUACGUACGG | 31 |
| (MHV) | | | ACGCCAAUCACUCUUAUA | |

To select a targeting sequence, one looks for a sequence that, when hybridized to a complementary sequence in the 3′-end region of the negative-strand RNA (SEQ ID NOS: 1-3), will be effective to disrupt stem-loop secondary structure in this region, and preferably, the initial stem structure in the region. By way of example, a suitable targeting sequence for the West Nile Virus (WNV in FIG. 4A) is a sequence that will disrupt the stem loop structure shown in the figure. Four general classes of sequences would be suitable (exemplary 12-14 base targeting sequences are shown for illustrative purposes):

(1) a sequence such as 5′AGTAGTTCGCCTGT3′ that targets the most 3′ bases of the stem and surrounding bases;

(2) a sequence such as 5′CTGACAAACTTA3′ that targets the complementary bases of the stem and surrounding bases;

(3) a sequence such as 5′TCGCCTGTGTGAGC 3′), that targets a portion of one or both “sides” of a stem and surrounding bases; typically, the sequence should disrupt at least all but 2-4 of the paired bases forming the stem structure;

(4) a sequence such as 5′AGTAGTTCAAACTT3′ that includes several (in this case, 5 complementary paired bases forming the stem, and optionally, adjacent bases on either side of the stem. The sense compound in this embodiment disrupts the stem structure by hybridizing to non-contiguous target sequences on opposite sides of the target secondary structure.

It will be appreciated how this selection procedure can be applied to the other sequences shown in Table 1. For example, for the yellow fever virus (YFV) shown in FIG. 4A, exemplary 12-14-base sequences patterned after the four general classes above, might include:

(1) a sequence such as 5′AGTAAATCCTGTG3′ that targets the most 3′ bases of the initial stem and surrounding bases;

(2) a sequence such as 5′CTGTGTGCTAATTG3′ that targets the complementary bases of the initial stem and surrounding bases;

(3) a sequence such as 5′AATCCTGTGTGCTAA3′), that targets a portion of both sides of a stem and surrounding bases;

(4) a sequence such as 5′AGTAAATCAATTGA3′ that includes several (in this case, all 4 complementary paired bases forming the stem, and optionally, adjacent bases on either side of the stem.

In addition, where the 3′-end region 40 bases include more than one stem structure, as in the case of the YFV, the targeting sequence can be selected to disrupt both structures, for example, with the 14-base targeting sequence 5′TAATTGAGGTGCAT3′ that extends across both stems in the virus region.

The latter approach is readily applied to other viruses that contain more than one predicted stem-loop secondary structure, such as the HCV sequence shown in FIG. 4A. Here one exemplary 14-base sequence capable of disrupting both stem structures would have the sequence: 5′TGGGGGCGACACTC3′.

It will be understood that targeting sequences so selected can be made shorter, e.g., 12 bases, or longer, e.g., 20 bases, and include a small number of mismatches, as long as the sequence is sufficiently complementary to disrupt the stem structure(s) upon hybridization with the target, and forms with the virus negative strand, a heteroduplex having a Tm of 45° C. or greater.

More generally, the degree of complementarity between the target and targeting sequence is sufficient to form a stable duplex. The region of complementarity of the sense oligomers with the target RNA sequence may be as short as 8-11 bases, but is preferably 12-15 bases or more, e.g. 12-20 bases, or 12-25 bases. A sense oligomer of about 14-15 bases is generally long enough to have a unique complementary sequence in the viral genome. In addition, a minimum length of complementary bases may be required to achieve the requisite binding Tₘ, as discussed below.

Oligomers as long as 40 bases may be suitable, where at least the minimum number of bases, e.g., 8-11, preferably 12-15 bases, are complementary to the target sequence. In general, however, facilitated or active uptake in cells is optimized at oligomer lengths less than about 30, preferably less than 25, and more preferably 20 or fewer bases. For PMO oligomers, described further below, an optimum balance of binding stability and uptake generally occurs at lengths of 13-18 bases.

The oligomer may be 100% complementary to the viral nucleic acid target sequence, or it may include mismatches, e.g., to accommodate variants, as long as a heteroduplex formed between the oligomer and viral nucleic acid target sequence is sufficiently stable to withstand the action of cellular nucleases and other modes of degradation which may occur in vivo. Oligomer backbones which are less susceptible to cleavage by nucleases are discussed below. Mismatches, if present, are less destabilizing toward the end regions of the hybrid duplex than in the middle. The number of mismatches allowed will depend on the length of the oligomer, the percentage of G:C base pairs in the duplex, and the position of the mismatch(es) in the duplex, according to well understood principles of duplex stability. Although such an antisense oligomer is not necessarily 100% complementary to the viral nucleic acid target sequence, it is effective to stably and specifically bind to the target sequence, such that a biological activity of the nucleic acid target, e.g., expression of viral protein(s), is modulated.

The stability of the duplex formed between the oligomer and the target sequence is a function of the binding Tₘ and the susceptibility of the duplex to cellular enzymatic cleavage. The Tₘ of an antisense compound with respect to complementary-sequence RNA may be measured by conventional methods, such as those described by Hames et al., Nucleic Acid Hybridization, IRL Press, 1985, pp. 107-108. Each sense oligomer should have a binding Tₘ, with respect to a complementary-sequence RNA, of greater than body temperature and preferably greater than 50° C. Tₘ's in the range 60-80° C. or greater are preferred. According to well known principles, the Tₘ of an oligomer compound, with respect to a complementary-based RNA hybrid, can be increased by increasing the ratio of C:G paired bases in the duplex, and/or by increasing the length (in base pairs) of the heteroduplex. At the same time, for purposes of optimizing cellular uptake, it may be advantageous to limit the size of the oligomer. For this reason, compounds that show high Tₘ (50° C. or greater) at a length of 20 bases or less are generally preferred over those requiring greater than 20 bases for high Tₘ values.

Tables 2 below shows exemplary targeting sequences, in a 5′-to-3′ orientation, that are complementary to upstream (3′-most sequence in the negative strand) and downstream portions of the 3′-40 base region of the negative strand of the viruses indicated. The sequence here provide a collection of sequences from which targeting sequences may be selected, according to the general sequence-selection rules discussed above.

TABLE 2

Exemplary Sense Sequences Targeting the 3′ End
Terminal Minus-Strand Stem Loops

| | | | | SEQ |
|---|---|---|---|---|
| | GenBank | | 3′ Sequences | ID |
| Virus | Acc. No. | Ncts. | (5′ to 3′) | NO. |
| St. Louis | M16614 | 1-20 | gnngatgttcgcgtcggtga | 32 |
| encephalitis | | 13-33 | gtcggtgagcggagaggaaac | 33 |
| Japanese | NC001437 | 1-20 | agaagtttatctgtgtg[aac | 34 |
| encephalitis | | 11-32 | ctct]gtgaacttcttggcttag | 35 |
| Murray Valley | NC 000943 | 1-20 | agacgttcatctgcgtgagc | 36 |
| encephalitis | | 5-25 | gttcatctgcgtgagcttccg | 37 |
| West Nile | NC 001563 | 1-22 | agtagttcgcctgtgtgagctg | 38 |
| | | 15-35 | gtgagctgacaaacttagtag | 39 |
| Yellow Fever | NC 002031 | 1-22 | agtaaatcctgtgtgctaattg | 40 |
| | | 13-31 | gtgctaattgaggtgcattg | 41 |
| Dengue - Type 2 | M20558 | 1-22 | agttgttagtctacgtggaccg | 42 |
| | | 12-32 | tacgtggaccgacaaagacag | 43 |
| Hepatitis C | NC 004102 | 1-16 | gccagccccctgatgg | 44 |
| | | 13-34 | atgggggcgacactccaccatg | 45 |
| Poliovirus- | NC 002058 | 1-20 | ttaaaacagctctggggttg | 46 |
| Mahoney | | 17-35 | gttgtacccaccccagagg | 47 |
| strain | | | | |
| Human | NC 001612 | 1-20 | ttaaaacagcctgtgggttg | 48 |
| enterovirus A | | 17-35 | gttgtacccacccacaggg | 49 |
| Human | NC 001472 | 1-20 | ttaaaacagcctgtgggttg | 50 |
| enterovirus B | | 17-34 | gttgttcccacccacagg | 51 |
| Human | NC 001428 | 1-20 | ttaaaacagctctggggttg | 52 |
| enterovirus C | | 17-35 | gttgctcccaccccagagg | 53 |
| Human | NC 001430 | 1-20 | ttaaaacagctctggggttg | 54 |
| enterovirus D | | 18-35 | ttgttcccaccccagagg | 55 |
| Human | NC 003988 | 1-20 | gagtgttcccacccaacagg | 56 |
| enterovirus E | | 15-34 | aacaggcccactgggtgttg | 57 |
| Bovine | NC 001859 | 1-20 | ttaaaacagcctgggggttg | 58 |
| enterovirus | | 17-35 | gttgtacccacccctgggg | 59 |
| Human | NC 001617 | 1-20 | ttaaaactgggagtgggttg | 60 |
| rhinovirus 89 | | 17-36 | gttgttcccactcactccac | 61 |
| Human | NC 001490 | 1-21 | ttaaaacagcggatgggtatc | 62 |
| rhinovirus B | | 12-31 | gatgggtatcccaccattcg | 63 |
| Foot-and-mouth | NC 004004 | 1-19 | ttgaaagggggcactaggg | 64 |
| disease | | 16-35 | agggtctcatctctagcacg | 65 |
| Hepatitis A | NC 001489 | 1-19 | ttcaagaggg gtctccggg | 66 |
| | | 19-39 | gaatttccggagtccctcttg | 67 |
| Feline | NC 001481 | 1-22 | gtaaaagaaatttgagacaatg | 68 |
| calicivirus | | 21-40 | gtctcaaactctgagcttc | 69 |
| Canine | NC 004542 | 1-21 | gttaatgagaaatggcttctg | 70 |
| calicivirus | | 16-37 | cttctgccatcgctctctcgag | 71 |
| Porcine | NC 000940 | 1-20 | gtgatcgtga tggctaattg | 72 |
| enteric | | 16-37 | aattgccgtccgttgcctattg | 73 |
| calicivirus | | | | |
| Calcivirus | NC 004064 | 1-23 | gtgatttaattatagagagatag | 74 |
| strain NB | | 10-31 | ttatagagagatagtgactttc | 75 |
| Norwalk | NC 001959 | 1-23 | gtgaatgatgatggcgtcaaaag | 76 |
| | | 18-38 | caaaagacgtcgttcctactg | 77 |
| Hepatitis E | NC 001434 | 1-18 | gccatggaggcccatcag | 78 |
| | | 14-35 | atcagtttattaaggctcctgg | 79 |
| Rubella | NC 001545 | 1-20 | atggaagctatcggacctcg | 80 |
| | | 9-30 | tatcggacctcgcttaggactc | 81 |
| SARS | NC 004718 | 1-23 | atattaggtttttacctacccag | 82 |
| coronavirus | | 18-38 | acccaggaaaagccaaccaac | 83 |
| TOR2 | | | | |
| Porcine | NC 003436 | 1-24 | acttaaaaagattttctatctacg | 84 |
| epidemic | | 12-29 | ttttctatctacgtacggatag | 85 |
| diarrhea | | | | |
| Transmissible | NC 002306 | 1-21 | acttttaaagtaaagtgagtg | 86 |
| gastroenteritis | | 10-29 | gtaaagtgagtggtagcgtgg | 87 |
| Bovine | NC 003045 | 1-22 | gattgcgagcgatttgcgtgcg | 88 |
| coronavirus | | 18-39 | gtgcgtgcatcccgcttcactg | 89 |
| Human | NC 002645 | 2-25 | cttaagtaccttatctatctac | 90 |
| coronavirus | | 19-37 | ag tctacagatagaaaagttg | 91 |
| 229E | | | | |
| Murine | NC 001846 | 1-21 | tataagagtgattggcgtccg | 92 |
| Hepatitis | | 18-39 | tccgtacgtaccctctcaactc | 93 |

### IV. Sense Oligonucleotide Analog Compounds

A. Properties

As detailed above, the sense oligonucleotide analog compound (the term “sense” indicates that the compound is targeted against the virus' antisense or negative-sense strand RNA has a base sequence targeting a region of the 3′ end 4 bases that are associated with secondary structure in the negative-strains RNA. In addition, the oligomer is able to effectively target infecting viruses, when administered to an infected host cell, e.g. in an infected mammalian subject. This requirement is met when the oligomer compound (a) has the ability to be actively taken up by mammalian cells, and (b) once taken up, form a duplex with the target ssRNA with a Tₘ greater than about 45° C.

As will be described below, the ability to be taken up by cells requires that the oligomer backbone be substantially uncharged, and, preferably, that the oligomer structure is recognized as, a substrate for active or facilitated transport across the cell membrane. The ability of the oligomer to form a stable duplex with the target RNA will also depend on the oligomer backbone, as well as factors noted above, the length and degree of complementarity of the sense oligomer with respect to the target, the ratio of G:C to A:T base matches, and the positions of any mismatched bases. The ability of the sense oligomer to resist cellular nucleases promotes survival and ultimate delivery of the agent to the cell cytoplasm.

Below are disclosed methods for testing any given, substantially uncharged backbone for its ability to meet these requirements.

A1. Active or Facilitated Uptake by Cells

The sense compound may be taken up by host cells by facilitated or active transport across the host cell membrane if administered in free (non-complexed) form, or by an endocytotic mechanism if administered in complexed form.

In the case where the agent is administered in free form, the sense compound should be substantially uncharged, meaning that a majority of its intersubunit linkages are uncharged at physiological pH. Experiments carried out in support of the invention indicate that a small number of net charges, e.g., 1-2 for a 15- to 20-mer oligomer, can in fact enhance cellular uptake of certain oligomers with substantially uncharged backbones. The charges may be carried on the oligomer itself, e.g., in the backbone linkages, or may be terminal charged-group appendages. Preferably, the number of charged linkages is no more than one charged linkage per four uncharged linkages. More preferably, the number is no more than one charged linkage per ten, or no more than one per twenty, uncharged linkages. In one embodiment, the oligomer is fully uncharged.

An oligomer may also contain both negatively and positively charged backbone linkages, as long as opposing charges are present in approximately equal number. Preferably, the oligomer does not include runs of more than 3-5 consecutive subunits of either charge. For example, the oligomer may have a given number of anionic linkages, e.g. phosphorothioate or N3′→P5′ phosphoramidate linkages, and a comparable number of cationic linkages, such as N,N-diethylenediamine phosphoramidates (Dagle, 2000). The net charge is preferably neutral or at most 1-2 net charges per oligomer.

In addition to being substantially or fully uncharged, the sense agent is preferably a substrate for a membrane transporter system (i.e. a membrane protein or proteins) capable of facilitating transport or actively transporting the oligomer across the cell membrane. This feature may be determined by one of a number of tests for oligomer interaction or cell uptake, as follows.

A first test assesses binding at cell surface receptors, by examining the ability of an oligomer compound to displace or be displaced by a selected charged oligomer, e.g., a phosphorothioate oligomer, on a cell surface. The cells are incubated with a given quantity of test oligomer, which is typically fluorescently labeled, at a final oligomer concentration of between about 10-300 nM. Shortly thereafter, e.g., 10-30 minutes (before significant internalization of the test oligomer can occur), the displacing compound is added, in incrementally increasing concentrations. If the test compound is able to bind to a cell surface receptor, the displacing compound will be observed to displace the test compound. If the displacing compound is shown to produce 50% displacement at a concentration of 10× the test compound concentration or less, the test compound is considered to bind at the same recognition site for the cell transport system as the displacing compound.

A second test measures cell transport, by examining the ability of the test compound to transport a labeled reporter, e.g., a fluorescence reporter, into cells. The cells are incubated in the presence of labeled test compound, added at a final concentration between about 10-300 nM. After incubation for 30-120 minutes, the cells are examined, e.g., by microscopy, for intracellular label. The presence of significant intracellular label is evidence that the test compound is transported by facilitated or active transport.

The sense compound may also be administered in complexed form, where the complexing agent is typically a polymer, e.g., a cationic lipid, polypeptide, or non-biological cationic polymer, having an opposite charge to any net charge on the sense compound. Methods of forming complexes, including bilayer complexes, between anionic oligonucleotides and cationic lipid or other polymer components, are well known. For example, the liposomal composition Lipofectin® (Felgner et al., 1987), containing the cationic lipid DOTMA (N-[1-(2,3-dioleyloxy)propyl]-N,N,N-trimethylammonium chloride) and the neutral phospholipid DOPE (dioleyl phosphatidyl ethanolamine), is widely used. After administration, the complex is taken up by cells through an endocytotic mechanism, typically involving particle encapsulation in endosomal bodies.

The sense compound may also be administered in conjugated form with an arginine-rich peptide linked to the 5′ or 3′ end of the antisense oligomer (see, for example, Moulton, Nelson, 2004). The peptide is typically 8-16 amino acids and consists of a mixture of arginine, and other amino acids including phenyalanine and cysteine. Exposure of cells to the peptide conjugated oligomer results in enhanced intracellular uptake and delivery to the RNA target.

Alternatively, and according to another aspect of the invention, the requisite properties of oligomers with any given backbone can be confirmed by a simple in vivo test, in which a labeled compound is administered to an animal, and a body fluid sample, taken from the animal several hours after the oligomer is administered, assayed for the presence of heteroduplex with target RNA. This method is detailed in subsection D below.

A2. Substantial Resistance to RNaseH

Two general mechanisms have been proposed to account for inhibition of expression by antisense oligonucleotides. (See e.g., Agrawal et al., 1990; Bonham et al., 1995; and Boudvillain et al., 1997). In the first, a heteroduplex formed between the oligonucleotide and the viral RNA acts as a substrate for RNaseH, leading to cleavage of the viral RNA. Oligonucleotides belonging, or proposed to belong, to this class include phosphorothioates, phosphotriesters, and phosphodiesters (unmodified “natural” oligonucleotides). Such compounds expose the viral RNA in an oligomer:RNA duplex structure to hydrolysis by RNaseH, and therefore loss of function.

A second class of oligonucleotide analogs, termed “steric blockers” or, alternatively, “RNaseH inactive” or “RNaseH resistant”, have not been observed to act as a substrate for RNaseH, and are believed to act by sterically blocking target RNA nucleocytoplasmic transport, splicing or translation. This class includes methylphosphonates (Toulme et al., 1996), morpholino oligonucleotides, peptide nucleic acids (PNA's), certain 2′-O-allyl or 2′-O-alkyl modified oligonucleotides (Bonham, 1995), and N3′→P5′ phosphoramidates (Gee, 1998; Ding, 1996).

A test oligomer can be assayed for its RNaseH resistance by forming an RNA:oligomer duplex with the test compound, then incubating the duplex with RNaseH under a standard assay conditions, as described in Stein et al. After exposure to RNaseH, the presence or absence of intact duplex can be monitored by gel electrophoresis or mass spectrometry.

A3. In Vivo Uptake

In accordance with another aspect of the invention, there is provided a simple, rapid test for confirming that a given sense oligomer type provides the required characteristics noted above, namely, high Tₘ, ability to be actively taken up by the host cells, and substantial resistance to RNaseH. This method is based on the discovery that a properly designed antisense compound will form a stable heteroduplex with the complementary portion of the viral RNA target when administered to a mammalian subject, and the heteroduplex subsequently appears in the urine (or other body fluid). Details of this method are also given in co-owned U.S. patent application Ser. No. 09/736,920, entitled “Non-Invasive Method for Detecting Target RNA” (Non-Invasive Method), the disclosure of which is incorporated herein by reference.

Briefly, a test oligomer containing a backbone to be evaluated, having a base sequence targeted against a known RNA, is injected into a mammalian subject. The sense oligomer may be directed against any intracellular RNA, including a host RNA or the RNA of an infecting virus. Several hours (typically 8-72) after administration, the urine is assayed for the presence of the sense-RNA heteroduplex. If heteroduplex is detected, the backbone is suitable for use in the sense oligomers of the present invention.

The test oligomer may be labeled, e.g. by a fluorescent or a radioactive tag, to facilitate subsequent analyses, if it is appropriate for the mammalian subject. The assay can be in any suitable solid-phase or fluid format. Generally, a solid-phase assay involves first binding the heteroduplex analyte to a solid-phase support, e.g., particles or a polymer or test-strip substrate, and detecting the presence/amount of heteroduplex bound. In a fluid-phase assay, the analyte sample is typically pretreated to remove interfering sample components. If the oligomer is labeled, the presence of the heteroduplex is confirmed by detecting the label tags. For non-labeled compounds, the heteroduplex may be detected by immunoassay if in solid phase format or by mass spectroscopy or other known methods if in solution or suspension format.

When the sense oligomer is complementary to a virus-specific region of the viral genome (such as 3′ end terminal region of the viral RNA, as described above), the method can be used to detect the presence of a given ssRNA virus, or reduction in the amount of virus during a treatment method.

B. Exemplary Oligomer Backbones

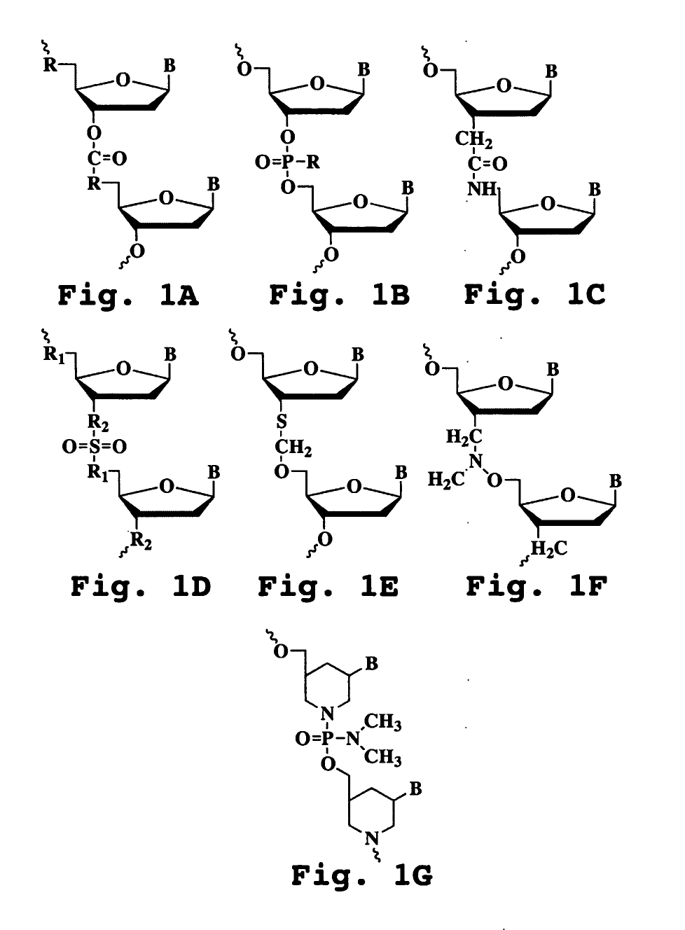

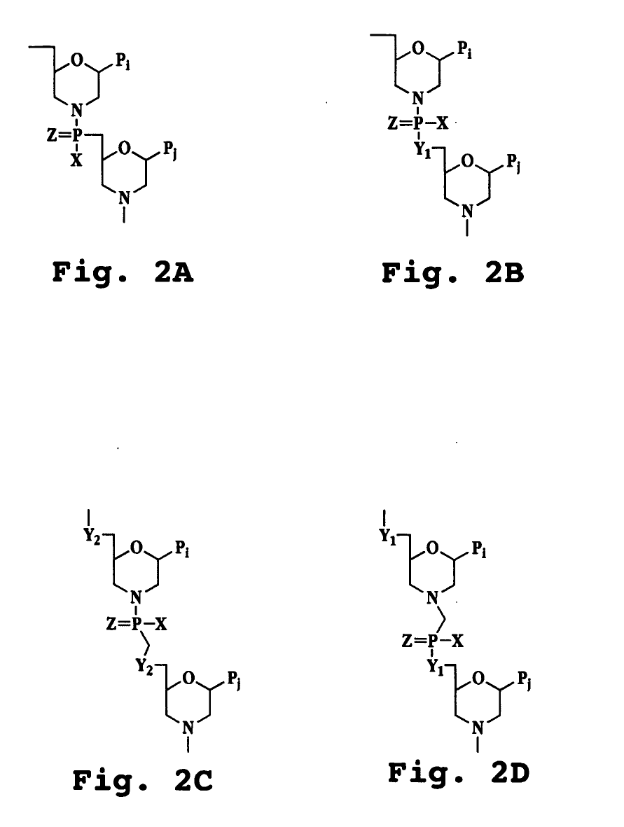

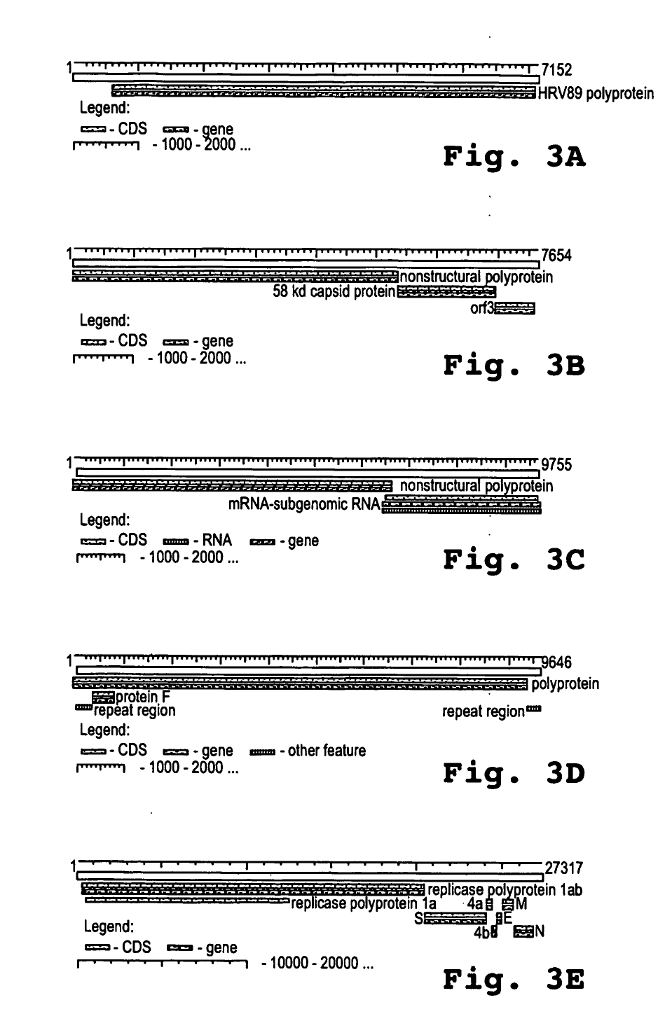

Examples of nonionic linkages that may be used in oligonucleotide analogs are shown in FIGS. 1A-1G. In these figures, B represents a purine or pyrimidine base-pairing moiety effective to bind, by base-specific hydrogen bonding, to a base in a polynucleotide, preferably selected from adenine, cytosine, guanine and uracil. Suitable backbone structures include carbonate (1A, R═O) and carbamate (1A, R═NH₂) linkages (Mertes and Coats 1969; Gait, Jones et al. 1974); alkyl phosphonate and phosphotriester linkages (1B, R=alkyl or —O-alkyl) (Lesnikowski, Jaworska et al. 1990); amide linkages (1C) (Blommers, Pieles et al. 1994); sulfone and sulfonamide linkages (1D, R₁, R₂═CH₂) (Roughten, 1995; McElroy, 1994); and a thioformacetyl linkage (1E) (Matteucci, 1990; Cross, 1997). The latter is reported to have enhanced duplex and triplex stability with respect to phosphorothioate antisense compounds (Cross, 1997). Also reported are the 3′-methylene-N-methylhydroxyamino compounds of structure 1F (Mohan, 1995).

Peptide nucleic acids (PNAs) (FIG. 1G) are analogs of DNA in which the backbone is structurally homomorphous with a deoxyribose backbone, consisting of N-(2-aminoethyl)glycine units to which pyrimidine or purine bases are attached. PNAs containing natural pyrimidine and purine bases hybridize to complementary oligonucleotides obeying Watson-Crick base-pairing rules, and mimic DNA in terms of base pair recognition (Egholm et al., 1993). The backbone of PNAs are formed by peptide bonds rather than phosphodiester bonds, making them well-suited for antisense applications. The backbone is uncharged, resulting in PNA/DNA or PNA/RNA duplexes which exhibit greater than normal thermal stability. PNAs are not recognized by nucleases or proteases.

A preferred oligomer structure employs morpholino-based subunits bearing base-pairing moieties, joined by uncharged linkages, as described above. Especially preferred is a substantially uncharged phosphorodiamidate-linked morpholino oligomer, such as illustrated in FIGS. 2A-2D. Morpholino oligonucleotides, including antisense oligomers, are detailed, for example, in co-owned U.S. Pat. Nos. 5,698,685, 5,217,866, 5,142,047, 5,034,506, 5,166,315, 5,185,444, 5,521,063, and 5,506,337, all of which are expressly incorporated by reference herein.

Important properties of the morpholino-based subunits include: the ability to be linked in a oligomeric form by stable, uncharged backbone linkages; the ability to support a nucleotide base (e.g. adenine, cytosine, guanine or uracil) such that the polymer formed can hybridize with a complementary-base target nucleic acid, including target RNA, with high Tₘ, even with oligomers as short as 10-14 bases; the ability of the oligomer to be actively transported into mammalian cells; and the ability of the oligomer:RNA heteroduplex to resist RNAse degradation.

Exemplary backbone structures for antisense oligonucleotides of the invention include the β-morpholino subunit types shown in FIGS. 2A-2D, each linked by an uncharged, phosphorus-containing subunit linkage. FIG. 2A shows a phosphorus-containing linkage which forms the five atom repeating-unit backbone, where the morpholino rings are linked by a 1-atom phosphoamide linkage. FIG. 2B shows a linkage which produces a 6-atom repeating-unit backbone. In this structure, the atom Y linking the 5′ morpholino carbon to the phosphorus group may be sulfur, nitrogen, carbon or, preferably, oxygen. The X moiety pendant from the phosphorus may be fluorine, an alkyl or substituted alkyl, an alkoxy or substituted alkoxy, a thioalkoxy or substituted thioalkoxy, or unsubstituted, monosubstituted, or disubstituted nitrogen, including cyclic structures, such as morpholines or piperidines. Alkyl, alkoxy and thioalkoxy preferably include 1-6 carbon atoms. The Z moieties are sulfur or oxygen, and are preferably oxygen.

The linkages shown in FIGS. 2C and 2D are designed for 7-atom unit-length backbones. In Structure 3C, the X moiety is as in Structure 3B, and the moiety Y may be methylene, sulfur, or, preferably, oxygen. In Structure 2D, the X and Y moieties are as in Structure 2B. Particularly preferred morpholino oligonucleotides include those composed of morpholino subunit structures of the form shown in FIG. 2B, where X═NH₂ or N(CH₃)₂, Y═O, and Z=O.

As noted above, the substantially uncharged oligomer may advantageously include a limited number of charged linkages, e.g. up to about 1 per every 5 uncharged linkages, more preferably up to about 1 per every 10 uncharged linkages. Therefore a small number of charged linkages, e.g. charged phosphoramidate or phosphorothioate, may also be incorporated into the oligomers.

The antisense compounds can be prepared by stepwise solid-phase synthesis, employing methods detailed in the references cited above. In some cases, it may be desirable to add additional chemical moieties to the antisense compound, e.g. to enhance pharmacokinetics or to facilitate capture or detection of the compound. Such a moiety may be covalently attached, typically to a terminus of the oligomer, according to standard synthetic methods. For example, addition of a polyethyleneglycol moiety or other hydrophilic polymer, e.g., one having 10-100 monomeric subunits, may be useful in enhancing solubility. One or more charged groups, e.g., anionic charged groups such as an organic acid, may enhance cell uptake. A reporter moiety, such as fluorescein or a radiolabeled group, may be attached for purposes of detection. Alternatively, the reporter label attached to the oligomer may be a ligand, such as an antigen or biotin, capable of binding a labeled antibody or streptavidin. In selecting a moiety for attachment or modification of an antisense oligomer, it is generally of course desirable to select chemical compounds of groups that are biocompatible and likely to be tolerated by a subject without undesirable side effects.

### V. Inhibition of Viral Replication

The sense compounds detailed above are useful in inhibiting replication of ssRNA viruses of the Flaviviridae, Picornoviridae, Caliciviridae, Togaviridae, Coronaviridae families and Hepatitis E virus. In one embodiment, such inhibition is effective in treating infection of a host animal by these viruses. Accordingly, the method comprises, in one embodiment, contacting a cell infected with the virus with a sense agent effective to inhibit the replication of the specific virus. In this embodiment, the sense agent is administered to a mammalian subject, e.g., human or domestic animal, infected with a given virus, in a suitable pharmaceutical carrier. It is contemplated that the sense oligonucleotide arrests the growth of the RNA virus in the host. The RNA virus may be decreased in number or eliminated with little or no detrimental effect on the normal growth or development of the host.

A. Identification of the Infective Agent

The specific virus causing the infection can be determined by methods known in the art, e.g. serological or cultural methods, or by methods employing the sense oligomers of the present invention.

Serological identification employs a viral sample or culture isolated from a biological specimen, e.g., stool, urine, cerebrospinal fluid, blood, etc., of the subject. Immunoassay for the detection of virus is generally carried out by methods routinely employed by those of skill in the art, e.g., ELISA or Western blot. In addition, monoclonal antibodies specific to particular viral strains or species are often commercially available.

Culture methods may be used to isolate and identify particular types of virus, by employing techniques including, but not limited to, comparing characteristics such as rates of growth and morphology under various culture conditions.

Another method for identifying the viral infective agent in an infected subject employs one or more sense oligomers targeting broad families and/or genera of viruses, e.g., Picornaviridae, Caliciviridae, Togaviridae and Flaviviridae. Sequences targeting any characteristic viral RNA can be used. The desired target sequences are preferably (i) common to broad virus families/genera, and (ii) not found in humans. Characteristic nucleic acid sequences for a large number of infectious viruses are available in public databases, and may serve as the basis for the design of specific oligomers.

For each plurality of oligomers, the following steps are carried out: (a) the oligomer(s) are administered to the subject; (b) at a selected time after said administering, a body fluid sample is obtained from the subject; and (c) the sample is assayed for the presence of a nuclease-resistant heteroduplex comprising the sense oligomer and a complementary portion of the viral genome. Steps (a)-(c) are carried for at least one such oligomer, or as many as is necessary to identify the virus or family of viruses. Oligomers can be administered and assayed sequentially or, more conveniently, concurrently. The virus is identified based on the presence (or absence) of a heteroduplex comprising the sense oligomer and a complementary portion of the viral genome of the given known virus or family of viruses.

Preferably, a first group of oligomers, targeting broad families, is utilized first, followed by selected oligomers complementary to specific genera and/or species and/or strains within the broad family/genus thereby identified. This second group of oligomers includes targeting sequences directed to specific genera and/or species and/or strains within a broad family/genus. Several different second oligomer collections, i.e. one for each broad virus family/genus tested in the first stage, are generally provided. Sequences are selected which are (i) specific for the individual genus/species/strains being tested and (ii) not found in humans.

B. Administration of the Sense Oligomer

Effective delivery of the sense oligomer to the target nucleic acid is an important aspect of treatment. In accordance with the invention, routes of sense oligomer delivery include, but are not limited to, various systemic routes, including oral and parenteral routes, e.g., intravenous, subcutaneous, intraperitoneal, and intramuscular, as well as inhalation, transdermal and topical delivery. The appropriate route may be determined by one of skill in the art, as appropriate to the condition of the subject under treatment. For example, an appropriate route for delivery of a sense oligomer in the treatment of a viral infection of the skin is topical delivery, while delivery of a sense oligomer for the treatment of a viral respiratory infection is by inhalation. The oligomer may also be delivered directly to the site of viral infection, or to the bloodstream.

The sense oligomer may be administered in any convenient vehicle which is physiologically acceptable. Such a composition may include any of a variety of standard pharmaceutically accepted carriers employed by those of ordinary skill in the art. Examples include, but are not limited to, saline, phosphate buffered saline (PBS), water, aqueous ethanol, emulsions, such as oil/water emulsions or triglyceride emulsions, tablets and capsules. The choice of suitable physiologically acceptable carrier will vary dependent upon the chosen mode of administration.

In some instances, liposomes may be employed to facilitate uptake of the sense oligonucleotide into cells. (See, e.g., Williams, S. A., Leukemia 10(12):1980-1989, 1996; Lappalainen et al., Antiviral Res. 23:119, 1994; Uhlmann et al., ANTISENSE OLIGONUCLEOTIDES: A NEW THERAPEUTIC PRINCIPLE, Chemical Reviews, Volume 90, No. 4, pages 544-584, 1990; Gregoriadis, G., Chapter 14, Liposomes, Drug Carriers in Biology and Medicine, pp. 287-341, Academic Press, 1979). Hydrogels may also be used as vehicles for sense oligomer administration, for example, as described in WO 93/01286. Alternatively, the oligonucleotides may be administered in microspheres or microparticles. (See, e.g., Wu, G. Y. and Wu, C. H., J. Biol. Chem. 262:4429-4432, 1987). Alternatively, the use of gas-filled microbubbles complexed with the antisense oligomers can enhance delivery to target tissues, as described in U.S. Pat. No. 6,245,747.

Sustained release compositions may also be used. These may include semipermeable polymeric matrices in the form of shaped articles such as films or microcapsules.

In one aspect of the method, the subject is a human subject, e.g., a patient diagnosed as having a localized or systemic viral infection. The condition of a patient may also dictate prophylactic administration of a sense oligomer of the invention, e.g. in the case of a patient who (1) is immunocompromised; (2) is a burn victim; (3) has an indwelling catheter; or (4) is about to undergo or has recently undergone surgery. In one preferred embodiment, the oligomer is a phosphorodiamidate morpholino oligomer, contained in a pharmaceutically acceptable carrier, and is delivered orally. In another preferred embodiment, the oligomer is a phosphorodiamidate morpholino oligomer, contained in a pharmaceutically acceptable carrier, and is delivered intravenously (IV).

In another application of the method, the subject is a livestock animal, e.g., a chicken, turkey, pig, cow or goat, etc, and the treatment is either prophylactic or therapeutic. The invention also includes a livestock and poultry food composition containing a food grain supplemented with a subtherapeutic amount of an antiviral sense compound of the type described above. Also contemplated is, in a method of feeding livestock and poultry with a food grain supplemented with subtherapeutic levels of an antiviral, an improvement in which the food grain is supplemented with a subtherapeutic amount of an antiviral oligonucleotide composition as described above.

The sense compound is generally administered in an amount and manner effective to result in a peak blood concentration of at least 200-400 nM sense oligomer. Typically, one or more doses of sense oligomer are administered, generally at regular intervals, for a period of about one to two weeks. Preferred doses for oral administration are from about 1-25 mg oligomer per 70 kg. In some cases, doses of greater than 25 mg oligomer/patient may be necessary. For IV administration, preferred doses are from about 0.5 mg to 10 mg oligomer per 70 kg. The sense oligomer may be administered at regular intervals for a short time period, e.g., daily for two weeks or less. However, in some cases the oligomer is administered intermittently over a longer period of time. Administration may be followed by, or concurrent with, administration of an antibiotic or other therapeutic treatment. The treatment regimen may be adjusted (dose, frequency, route, etc.) as indicated, based on the results of immunoassays, other biochemical tests and physiological examination of the subject under treatment.

C. Monitoring of Treatment

An effective in vivo treatment regimen using the sense oligonucleotides of the invention may vary according to the duration, dose, frequency and route of administration, as well as the condition of the subject under treatment (i.e., prophylactic administration versus administration in response to localized or systemic infection). Accordingly, such in vivo therapy will often require monitoring by tests appropriate to the particular type of viral infection under treatment, and corresponding adjustments in the dose or treatment regimen, in order to achieve an optimal therapeutic outcome. Treatment may be monitored, e.g., by general indicators of infection, such as complete blood count (CBC), nucleic acid detection methods, immunodiagnostic tests, viral culture, or detection of heteroduplex.

The efficacy of an in vivo administered sense oligomer of the invention in inhibiting or eliminating the growth of one or more types of RNA virus may be determined from biological samples (tissue, blood, urine etc.) taken from a subject prior to, during and subsequent to administration of the sense oligomer. Assays of such samples include (1) monitoring the presence or absence of heteroduplex formation with target and non-target sequences, using procedures known to those skilled in the art, e.g., an electrophoretic gel mobility assay; (2) monitoring the amount of viral protein production, as determined by standard techniques such as ELISA or Western blotting, or (3) measuring the effect on viral titer, e.g. by the method of Spearman-Karber. (See, for example, Pari, G. S. et al., Antimicrob. Agents and Chemotherapy 39(5):1157-1161, 1995; Anderson, K. P. et al., Antimicrob. Agents and Chemotherapy 40(9):2004-2011, 1996, Cottral, G. E. (ed) in: Manual of Standard Methods for Veterinary Microbiology, pp. 60-93, 1978).

A preferred method of monitoring the efficacy of the sense oligomer treatment is by detection of the sense-RNA heteroduplex. At selected time(s) after sense oligomer administration, a body fluid is collected for detecting the presence and/or measuring the level of heteroduplex species in the sample. Typically, the body fluid sample is collected 3-24 hours after administration, preferably about 6-24 hours after administering. As indicated above, the body fluid sample may be urine, saliva, plasma, blood, spinal fluid, or other liquid sample of biological origin, and may include cells or cell fragments suspended therein, or the liquid medium and its solutes. The amount of sample collected is typically in the 0.1 to 10 ml range, preferably about 1 ml of less.

The sample may be treated to remove unwanted components and/or to treat the heteroduplex species in the sample to remove unwanted ssRNA overhang regions, e.g. by treatment with RNase. It is, of course, particularly important to remove overhang where heteroduplex detection relies on size separation, e.g., electrophoresis of mass spectroscopy.

A variety of methods are available for removing unwanted components from the sample. For example, since the heteroduplex has a net negative charge, electrophoretic or ion exchange techniques can be used to separate the heteroduplex from neutral or positively charged material. The sample may also be contacted with a solid support having a surface-bound antibody or other agent specifically able to bind the heteroduplex. After washing the support to remove unbound material, the heteroduplex can be released in substantially purified form for further analysis, e.g., by electrophoresis, mass spectroscopy or immunoassay.

### EXAMPLES

The following examples illustrate but are not intended in any way to limit the invention.

### Materials and Methods

Standard recombinant DNA techniques were employed in all constructions, as described in Ausubel, F M et al., in CURRENT PROTOCOLS IN MOLECULAR BIOLOGY, John Wiley and Sons, Inc., Media, Pa., 1992 and Sambrook, J. et al., in MOLECULAR CLONING: A LABORATORY MANUAL, Cold Spring Harbor Laboratory Press, Cold Spring Harbor, N.Y., Vol. 2, 1989).

### Example 1

### Sense Inhibition of Flaviviridae (Hepatitis C Virus) In Vitro

The inhibitory effect on Hepatitis C virus (HCV) of a phosphorodiamidate morpholino oligomer (PMO) having a sequence targeted to the 3′ end terminus of Hepatitis C Virus was evaluated. The phosphorodiamidate morpholino oligomers (PMO) were synthesized at AVI BioPharma (Corvallis, Oreg.), as described in Summerton and Weller, 1997. Purity of the full-length oligomer was greater than 90% as determined by reverse-phase high-pressure liquid chromatography and MALDI TOF mass spectroscopy. The lyophilized PMOs were dissolved in sterile 0.9% NaCl and filtered through 0.2 μm Acrodisc filters (Gelman Sciences, Ann Arbor, Mich.) prior to use in cell cultures.

The PMO includes a nucleic acid sequence targeting the 3′ terminal end of the HCV minus-strand RNA. The target sequence (GenBank NC 004102 1-16; SEQ ID NO: 7) and targeting sequence (SEQ ID NO: 44) are as follows:

| | |
|---|---|
| 3′ end (-strand) HCV: | |
| 3′-CGGUCGGGGGACUACCAGUGUC . . . | SEQ ID NO: 7 |
| HCV sense PMO: | |
| 5′-GCCAGCCCCCTGATGG-3′ | SEQ ID NO: 44 |

Figure Z shows the position of the HCV sense PMO relative to the 5′ end of HCV RNA sequence.

A human cell line, FLC4, was infected with Hepatitis C virus and, six days post infection, treated with the HCV sense PMO (SEQ ID NO: 7) or a scramble control sequence (5′-CGCGACCCCTGCGATG-3′) at 40 ug/ml for 24 hours. Treated cells were harvested on day seven and nucleic acid extracts prepared according to standard techniques. A PCR-based assay that detects HCV-specific RNA was performed and the results are shown in FIG. 5. Compared to the infected serum positive control (lanes 3 & 4) and the scramble control PMO (lanes 7 & 8) the sense PMO (SEQ ID NO: 44, lanes 5 & 6) indicated a substantial reduction in viral protein expression. Lanes 1 & 2 are cells treated with normal human serum and act as a negative control.

### Example 2

### Antisense PMO Reduction of MHV Cytopathic Effects In Vitro

The observation of cytopathic effects (CPE) is a visual measure of antiviral drug activity. This example demonstrates the antiviral activity of a sense antiviral PMO targeted to the 3′ terminal end of the negative strand of the coronovirus murine hepatitis virus (MHV) in an assay designed to measure CPE. Vero-E6 cells were cultured in DMEM with 10% fetal bovine serum. Vero-E6 cells were plated at approximately 75% confluence in replicate 25 cm² culture flasks. Cells were rinsed and incubated in 1 ml of complete VP-SFM (virus production serum-free medium, Invitrogen) containing the specified concentration of sense antiviral PMO-P003 conjugate (5TERM-neg PMO, SEQ ID NO:92) or a PMO-P003 conjugate with an irrelevant sequence (DSCR, 5′-AGTCTCGACTTGCTACCTCA-3′) for 12-16 h (overnight). The arginine-rich peptide P003 (R₉F₂C-5′-PMO) was conjugated to the 5′ terminus of both PMOs and facilitated uptake into tissue culture cells as described previously (Moulton, Nelson et al. 2004). Vero-E6 cells were pretreated with PMO at either 20 or 3 μM, inoculated with SARS-CoV at a multiplicity of approximately 0.1 PFU/cell by adding virus directly to the treatment medium for 1 h at 37 C and cultured in the presence of 5TERM-neg PMO (SEQ ID NO:92) or DSCR at either 20 or 3 μM. After 24 h, the medium was replaced by fresh complete VP-SFM and cells were incubated an additional 24 h at 37 C. All cell cultures were incubated in the presence of 5% CO₂. 48 h after inoculation, the cells were fixed, decontaminated and stained with 0.1% crystal violet. CPE is visualized by phase contrast microscopy and recorded with a digital camera as shown in FIG. 6. The data for the 5TERM-neg treatment correspond to SEQ ID NO:92. From the data presented in FIG. 6, it is clear that the 5TERM-neg PMO prevented MHV-induced CPE at concentrations as low as 3 micromolar when compared to the DSCR control PMO.

From the foregoing, it will be appreciated how various objects and features of the invention are met. The sense oligonucleotide analog compound, by targeting the antisense or negative-strand of the RNA with a sense oligonucleotide analog, inhibits viral replication by inhibiting synthesis of viral mRNA needed for production of viral protein. This is an efficient targeting mechanism, since RNA replication to produce sense-strand RNA strand appears to be a much more active (measured by relative numbers of positive and negative strand viral RNA) replication event than replication to produce the intermediate negative-strand RNA.

The analog is stable in the body and for some analog structures, e.g., PMO, may be administered orally. Further, the formation of heteroduplexes between the analog and viral target may be used to confirm the presence or absence of infection by a flavivirus, and/or the confirm uptake of the therapeutic agent by the host.

TABLE 3
Sequence Listing Table

| | |
|---|---|
| SEQ ID | |
| NO. | Sequence, 5′ to 3′ |

| | |
|---|---|
| 1 | GAAAUCUGUUUCCUCUCCGCUCACCGACGCGAACAUNNNC |
| 2 | CAACGAUACUAAGCCAAGAAGUUCACACAGAUAAACUUCU |
| 3 | AAACAAUACUGAGAUCGGAAGCUCACGCAGAUGAACGUCU |
| 4 | AAACACUACUAAGUUUGUCAGCUCACACAGGCGAACUACU |
| 5 | UUGCAGACCAAUGCACCUCAAUUAGCACACAGGAUUUACU |
| 6 | CAAAGAAUCUGUCUUUGUCGGUCCACGUAGACUAACAACU |
| 7 | GUGAUUCAUGGUGGAGUGUCGCCCCCAUCAGGGGGCUGGC |
| 8 | GUGGGCCUCUGGGGUGGGUACAACCCCAGAGCUGUUUUAA |
| 9 | GUGGGCCCUGUGGGUGGGUACAACCCACAGGCUGUUUUAA |
| 10 | AAUGGGCCUGUGGGUGGGAACAACCCACAGGCUGUUUUAA |
| 11 | GUGGGCCUCUGGGGUGGGAGCAACCCCAGAGCUGUUUUAA |
| 12 | GUGGGCCUCUGGGGUGGGAACAACCCCAGAGCUGUUUUAA |
| 13 | AGAGUACAACACCCAGUGGGCCUGUUGGGUGGGAACACUC |
| 14 | GUGGGCCCCAGGGGUGGGUACAACCCCCAGGCUGUUUUAA |
| 15 | AUGGGUGGAGUGAGUGGGAACAACCCACUCCCAGUUUUAA |
| 16 | CCAAUGGGUCGAAUGGUGGGAUACCCAUCCGCUGUUUUAA |
| 17 | GUUGGCGUGCUAGAGAUGAGACCCUAGUGCCCCCUUUCAA |
| 18 | CCAAGAGGGACUCCGGAAAUUCCCGGAGACCCCUCUUGAA |
| 19 | GAAGCUCAGAGUUUGAGACAUUGUCUCAAAUUUCUUUUAC |
| 20 | GAGCUCGAGAGAGCGAUGGCAGAAGCCAUUUCUCAUUAAC |
| 21 | GCCCAAUAGGCAACGGACGGCAAUUAGCCAUCACGAUCAC |
| 22 | AAGAAAAGUGAAAGUCACUAUCUCUCUAUAAUUAAAUCAC |
| 23 | AGCAGUAGGAACGACGUCUUUUGACGCCAUCAUCAUUCAC |
| 24 | UGAUGCCAGGAGCCUUAAUAAACUGAUGGGCCUCCAUGGC |
| 25 | AUGGGAAUGGGAGUCCUAAGCGAGGUCCGAUAGCUUCCAU |
| 26 | AGGUUGGUUGGCUUUUCCUGGGUAGGUAAAAACCUAAUAU |
| 27 | AAAAGAGCUAACUAUCCGUAGAUAGAAAAUCUUUUUAAGU |
| 28 | AAGAGAUAUAGCCACGCUACACUCACUUUACUUUAAAAGU |
| 29 | UCAGUGAAGCGGGAUGCACGCACGCAAAUCGCUCGCAAUC |
| 30 | AAGCAACUUUUCUAUCUGUAGAUAGAUAAGGUACUUAAGU |
| 31 | AGAGUUGAGAGGGUACGUACGGACGCCAAUCACUCUUAUA |
| 32 | GNNGATGTTCGCGTCGGTGA |
| 33 | GTCGGTGAGCGGAGAGGAAAC |
| 34 | AGAAGTTTATCTGTGTGAAC |
| 35 | CTGTGTGAACTTCTTGGCTTAG |
| 36 | AGACGTTCATCTGCGTGAGC |
| 37 | GTTCATCTGCGTGAGCTTCCG |
| 38 | AGTAGTTCGCCTGTGTGAGCTG |
| 39 | GTGAGCTGACAAACTTAGTAG |
| 40 | AGTAAATCCTGTGTGCTAATTG |
| 41 | GTGCTAATTGAGGTGCATTG |
| 42 | AGTTGTTAGTCTACGTGGACCG |
| 43 | TACGTGGACCGACAAAGACAG |
| 44 | GCCAGCCCCCTGATGG |
| 45 | ATGGGGGCGACACTCCACCATG |
| 46 | TTAAAACAGCTCTGGGGTTG |
| 47 | GTTGTACCCACCCCAGAGG |
| 48 | TTAAAACAGCCTGTGGGTTG |
| 49 | GTTGTACCCACCCACAGGG |
| 50 | TTAAAACAGCCTGTGGGTTG |
| 51 | GTTGTTCCCACCCACAGG |
| 52 | TTAAAACAGCTCTGGGGTTG |
| 53 | GTTGCTCCCACCCCAGAGG |
| 54 | TTAAAACAGCTCTGGGGTTG |
| 55 | TTGTTCCCACCCCAGAGG |
| 56 | GAGTGTTCCCACCCAACAGG |
| 57 | AACAGGCCCACTGGGTGTTG |
| 58 | TTAAAACAGCCTGGGGGTTG |
| 59 | GTTGTACCCACCCCTGGGG |
| 60 | TTAAAACTGGGAGTGGGTTG |
| 61 | GTTGTTCCCACTCACTCCAC |
| 62 | TTAAAACAGCGGATGGGTATC |
| 63 | GATGGGTATCCCACCATTCG |
| 64 | TTGAAAGGGGGCACTAGGG |
| 65 | AGGGTCTCATCTCTAGCACG |
| 66 | TTCAAGAGGG GTCTCCGGG |
| 67 | GAATTTCCGGAGTCCCTCTTG |
| 68 | GTAAAAGAAATTTGAGACAATG |
| 69 | GTCTCAAACTCTGAGCTTC |
| 70 | GTTAATGAGAAATGGCTTCTG |
| 71 | CTTCTGCCATCGCTCTCTCGAG |
| 72 | GTGATCGTGA TGGCTAATTG |
| 73 | AATTGCCGTCCGTTGCCTATTG |
| 74 | GTGATTTAATTATAGAGAGATAG |
| 75 | TTATAGAGAGATAGTGACTTTC |
| 76 | GTGAATGATGATGGCGTCAAAAG |
| 77 | CAAAAGACGTCGTTCCTACTG |
| 78 | GCCATGGAGGCCCATCAG |
| 79 | ATCAGTTTATTAAGGCTCCTGG |
| 80 | ATGGAAGCTATCGGACCTCG |
| 81 | TATCGGACCTCGCTTAGGACTC |
| 82 | ATATTAGGTTTTTACCTACCCAG |
| 83 | ACCCAGGAAAAGCCAACCAAC |
| 84 | ACTTAAAAAGATTTTCTATCTACG |
| 85 | TTTTCTATCTACGTACGGATAG |
| 86 | ACTTTTAAAGTAAAGTGAGTG |
| 87 | GTAAAGTGAGTGGTAGCGTGG |
| 88 | GATTGCGAGCGATTTGCGTGCG |
| 89 | GTGCGTGCATCCCGCTTCACTG |
| 90 | CTTAAGTACCTTATCTATCTACAG |
| 91 | TCTACAGATAGAAAAGTTG |
| 92 | TATAAGAGTGATTGGCGTCCG |
| 93 | TCCGTACGTACCCTCTCAACTC |

It is claimed:

1. An oligonucleotide analog compound for use in inhibiting replication in mammalian host cells of an RNA virus having a single-stranded, positive-sense RNA genome and selected from the Flaviviridae, Picornoviridae, Caliciviridae, Togaviridae, or Coronaviridae families and hepatitis E virus, and characterized by:
(i) a nuclease-resistant backbone,
(ii) capable of uptake by mammalian host cells,
(iii) containing between 12-40 nucleotide bases,
(iv) having a targeting sequence of at least 12 subunits that is complementary to a region associated with stem-loop secondary structure within the 3′-terminal end 40 bases of the negative-sense RNA strand of the virus, and
(v) capable of forming with the negative-strand viral ssRNA genome, a heteroduplex structure having a Tm of dissociation of at least 45° C. and disruption of said stem-loop secondary structure.

2. The compound of claim 1, composed of morpholino subunits linked by uncharged, phosphorus-containing intersubunit linkages, joining a morpholino nitrogen of one subunit to a 5′ exocyclic carbon of an adjacent subunit.

3. The compound of claim 2, wherein said intersubunit linkages are phosphorodiamidate linkages.

4. The compound of claim 3, wherein said morpholino subunits are joined by phosphorodiamidate linkages, in accordance with the structure: 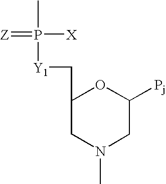
where Y₁═O, Z=O, Pj is a purine or pyrimidine base-pairing moiety effective to bind, by base-specific hydrogen bonding, to a base in a polynucleotide, and X is alkyl, alkoxy, thioalkoxy, or alkyl amino.

5. The compound of claim 4, wherein X═NR₂, where each R is independently hydrogen or methyl.

6. The compound of claim 2, wherein said oligomer has a Tₘ, with respect to binding to said viral target sequence, of greater than about 50° C., and said compound is actively taken up by mammalian cells.

7. The compound of claim 1, wherein said targeting sequence is complementary to a region associated with stem-loop secondary structure within the sequence selected from the group consisting of:
(i) SEQ ID NO. 1, for St Louis encephalitis virus;
(ii) SEQ ID NO. 2, for Japanese encephalitis virus;
(iii) SEQ ID NO. 3, for a Murray Valley encephalitis virus;
(iv) SEQ ID NO. 4, for a West Nile fever virus;
(v) SEQ ID NO. 5, for a Yellow fever virus
(vi) SEQ ID NO. 6, for a Dengue type 2 virus; and
(vi) SEQ ID NO. 7, for a Hepatitis C virus.

8. The compound of claim 1, directed against a member of the Picornaviridae, wherein said targeting sequence is complementary to a region associated with stem-loop secondary structure within the sequence selected from the group consisting of:
(i) SEQ ID NO. 8, for a polio virus of the Mahoney and Sabin strains;
(ii) SEQ ID NO. 9, for a Human enterovirus A;
(iii) SEQ ID NO. 10, for a Human enterovirus B;
(iv) SEQ ID NO. 11, for a Human enterovirus C;
(v) SEQ ID NO. 12, for a Human enterovirus D;
(vi) SEQ ID NO. 13, for a Human enterovirus E;
(vii) SEQ ID NO. 14, for a Bovine enterovirus;
(viii) SEQ ID NO. 15, for Human rhinovirus 89;
(ix) SEQ ID NO. 16, for Human rhinovirus B;
(x) SEQ ID NO. 17, for Foot-and-mouth disease virus; and
(xi) SEQ ID NO. 18, for a hepatitis A virus,

9. The compound of claim 1, directed against member of the Caliciviridae, wherein said targeting sequence is complementary to a region associated with stem-loop secondary structure within the sequence selected from the group consisting of:
(i) SEQ ID NO. 19, for Feline Calicivirus;
(ii) SEQ ID NO. 20, for Canine Calicivirus;
(iii) SEQ ID NO. 21, for Porcine enteric calicivirus;
(iv) SEQ ID NO. 22, for Calicivirus strain NB; and
(v) SEQ ID NO. 23, for Norwalk virus.

10. The compound of claim 1, directed against Hepatitis E virus, wherein said targeting sequence is complementary to a region associated with stem-loop secondary structure within the sequence identified as SEQ ID NO: 24.

11. The compound of claim 1, directed against a member of the Togaviridae, Rubella virus, wherein said targeting sequence is complementary to a region associated with stem-loop secondary structure within the sequence identified as SEQ ID NO: 25.

12. The compound of claim 1, directed against member of the Coronaviridae, wherein said targeting sequence is complementary to a region associated with stem-loop secondary structure within the sequence selected from the group consisting of:
(i) SEQ ID NO. 26, for SARS coronavirus TOR2;
(ii) SEQ ID NO. 27, for Porcine epidemic diarrhea virus;
(iii) SEQ ID NO. 28, for Transmissible gastroenteritis virus;
(iv) SEQ ID NO. 29, for Bovine coronavirus;
(v) SEQ ID NO. 30, for Human coronavirus 229E. and
(vi) SEQ ID NO. 31, for Murine hepatitis virus.

13. The compound of claim 1, complexed with a complementary-sequence at the 3′-end region of the negative-strand RNA of the virus.

14. A method of inhibiting, in a mammalian host cell, replication of an RNA virus from the Flaviviridae, Picornoviridae, Caliciviridae, Togaviridae, Coronaviridae families and hepatitis E virus, said virus having a single-stranded, positive-sense genome, said method comprising
(a) exposing the host cells to an oligonucleotide analog compound characterized by:
(i) a nuclease-resistant backbone,
(ii) capable of uptake by mammalian host cells,
(iii) containing between 12-40 nucleotide bases, and
(iv) having a targeting sequence of at least 12 subunits that is complementary to a region associated with stem-loop secondary structure within the 3′-terminal end 40 bases of the negative-sense RNA strand of the virus, and
(b) by said exposing, forming within said cells a heteroduplex structure composed of the negative sense strand of the virus and the oligonucleotide compound, and characterized by a Tm of dissociation of at least 45° C. and disruption of said stem-loop secondary structure.

15. The method of claim 14, wherein said oligonucleotide is administered to a mammalian subject infected with said virus, or at risk of infection with said virus.

16. The method of claim 15, wherein said oligonucleotide is composed of morpholino subunits linked by uncharged, phosphorus-containing intersubunit linkages, joining a morpholino nitrogen of one subunit to a 5′ exocyclic carbon of an adjacent subunit.

17. The method of claim 16, wherein said intersubunit linkages are phosphorodiamidate linkages.

18. The method of claim 17, wherein said morpholino subunits are joined by phosphorodiamidate linkages, in accordance with the structure: 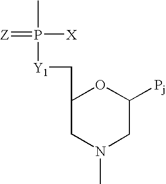
where Y₁=O, Z=O, Pj is a purine or pyrimidine base-pairing moiety effective to bind, by base-specific hydrogen bonding, to a base in a polynucleotide, and X is alkyl, alkoxy, thioalkoxy, or alkyl amino.

19. The method of claim 18, wherein X═NR₂, where each R is independently hydrogen or methyl.

20. The method of claim 17, wherein said compound is administered orally to a mammalian subject infected with the virus or at risk of infection with the virus.

21. The compound of claim 14, wherein said targeting sequence is complementary to a region associated with stem-loop secondary structure within the sequence selected from the group consisting of:
(i) SEQ ID NO. 1, for St Louis encephalitis virus;
(ii) SEQ ID NO. 2, for Japanese encephalitis virus;
(iii) SEQ ID NO. 3, for a Murray Valley encephalitis virus;
(iv) SEQ ID NO. 4, for a West Nile fever virus;
(v) SEQ ID NO. 5, for a Yellow fever virus
(vi) SEQ ID NO. 6, for a Dengue type 2 virus; and
(vii) SEQ ID NO. 7, for a Hepatitis C virus.

22. The method of claim 14, directed against a member of the Picornaviridae, wherein said targeting sequence is complementary to a region associated with stem-loop secondary structure within the sequence selected from the group consisting of:
(i) SEQ ID NO. 8, for a polio virus of the Mahoney and Sabin strains;
(ii) SEQ ID NO. 9, for a Human enterovirus A;
(iii) SEQ ID NO. 10, for a Human enterovirus B;
(iv) SEQ ID NO. 11, for a Human enterovirus C;
(v) SEQ ID NO. 12, for a Human enterovirus D;
(vi) SEQ ID NO. 13, for a Human enterovirus E;
(vii) SEQ ID NO. 14, for a Bovine enterovirus;
(viii) SEQ ID NO. 15, for Human rhinovirus 89;
(ix) SEQ ID NO. 16, for Human rhinovirus B;
(x) SEQ ID NO. 17, for Foot-and-mouth disease virus; and
(xi) SEQ ID NO. 18, for a hepatitis A virus,

23. The method of claim 14, directed against member of the Caliciviridae, wherein said targeting sequence is complementary to a region associated with stem-loop secondary structure within the sequence selected from the group consisting of:
(i) SEQ ID NO. 19, for Feline Calicivirus;
(ii) SEQ ID NO. 20, for Canine Calicivirus;
(iii) SEQ ID NO. 21, for Porcine enteric calicivirus;
(iv) SEQ ID NO. 22, for Calicivirus strain NB; and
(v) SEQ ID NO. 23, for Norwalk virus.

24. The method of claim 14, directed against Hepatitis E virus, wherein said targeting sequence is complementary to a region associated with stem-loop secondary structure within the sequence identified as SEQ ID NO: 24.

25. The method of claim 14, directed against a member of the Togaviridae, Rubella virus, wherein said targeting sequence is complementary to a region associated with stem-loop secondary structure within the sequence identified as SEQ ID NO: 25.

26. The method of claim 13, directed against member of the Coronaviridae, wherein said targeting sequence is complementary to a region associated with stem-loop secondary structure within the sequence selected from the group consisting of:
(i) SEQ ID NO. 26, for SARS coronavirus TOR2;
(ii) SEQ ID NO. 27, for Porcine epidemic diarrhea virus;
(iii) SEQ ID NO. 28, for Transmissible gastroenteritis virus;
(iv) SEQ ID NO. 29, for Bovine coronavirus;
(v) SEQ ID NO. 30, for Human coronavirus 229E. and
(vi) SEQ ID NO. 31, for Murine hepatitis virus.

27. A method of confirming the presence of an effective interaction between a picornavirus, calicivirus, togavirus, coronavirus, hepatitis E virus, or flavivirus infecting a mammalian subject, and an uncharged morpholino sense oligonucleotide analog compound against the infecting virus, comprising
(a) administering said compound to the subject, where said compound has (a) a sequence of 12-40 subunits, including a targeting sequence of at least 12 subunits that is complementary to a region associated with stem-loop secondary structure within the 3′-terminal end 40 bases of the negative-sense RNA strand of the virus, (b) morpholino subunits linked by uncharged, phosphorus-containing intersubunit linkages, each linkage joining a morpholino nitrogen of one subunit to a 5′ exocyclic carbon of an adjacent subunit, and (c) is capable of forming with the negative-strand viral ssRNA genome, a heteroduplex structure characterized by a Tm of dissociation of at least 45° C. and disruption of said stem-loop secondary structure,
(b) at a selected time after said administering, obtaining a sample of a body fluid from the subject; and
(c) assaying the sample for the presence of a nuclease-resistant heteroduplex comprising the sense oligonucleotide complexed with a complementary-sequence 3′-end region of the negative-strand RNA of the virus.

28. The method of claim 27, wherein the linkages are phosphorodiamidate linkages.

29. The method of claim 27, for use in determining the effectiveness of treating a picornavirus, calicivirus, togavirus, coronavirus, hepatitis E virus or flavivirus infection by administering said oligomer, wherein said administering, obtaining, and assaying is conducted at periodic intervals throughout a treatment period.
